(19) United States
(12) Patent Application Publication
(10) Pub. No.: US 2002/0068358 A1
(43) Pub. Date: Jun. 6, 2002

(54) In vitro embryo culture device

(76) Inventors: Michael J. Campbell, Louisville, KY (US); K.C. Fadem, Atlanta, GA (US)

Correspondence Address: DINSMORE & SHOHL, LLP, 1900 CHEMED CENTER, 255 EAST FIFTH STREET, CINCINNATI, OH, 45202, US

(21) Appl. No.: 09/827,301

(22) Filed: Apr. 5, 2001

Related U.S. Application Data
(63) Continuation-in-part of application No. 09/450,963, filed on Nov. 30, 1999.
(63) Continuation-in-part of application No. 09/067,715, filed on Apr. 28, 1998, now abandoned.
(60) Provisional application No. 60/242,238, filed on Oct. 20, 2000.

(51) Int. Cl. C12M 1/00

(52) U.S. Cl. 435/289.1

(57) ABSTRACT

An embryo support assembly, embryo support system, a method for maintaining the viability of a growing embryo, and a method for shipping a metabolically active embryo are provided.

This application claims the benefit of application serial No. 60/242,238 (filed Oct. 20, 2000), and is a continuation-in-part of application Ser. No. 09/450,963 (filed Nov. 30, 1999), which is a continuation-in-part of application Ser. No. 09/067,715 (filed on Apr. 28, 1998, and now abandoned). All of the above-referenced applications are incorporated herein by way of reference.

### TECHNICAL FIELD OF THE INVENTION

The present invention relates to apparatus and methods for growing embryos in vitro.

### BACKGROUND OF THE INVENTION

Clinical biologists and embryologists have been growing embryos in vitro for over 20 years. Amazingly, they still utilize essentially the same procedure that was first reported in 1978: an egg is collected and combined with the appropriate genetic material (e.g., sperm if the egg is to be fertilized, or the nucleus of a donor cell if a cloning process is used); the fertilized/cloned egg is placed in a glass petri dish with a nutritional “soup” or embryo growth media; the petri dish is placed into an incubation chamber for 3-7 days until the “embryo” has reached the proper stage of development; the mature embryo is then retrieved and transferred into the mother's uterus or the desired inner cellular material is harvested (e.g., stem cells). This process is very labor intensive and must be conducted by highly specialized personnel in well-equipped, expensive laboratories.

Embryos are grown in vitro for a variety of reasons. For example, many women are unable to become pregnant naturally due to any number of medical reasons (e.g., occlusion or dysfunction of the fallopian tubes). In such instances, in vitro fertilization (“IVF”) technology may be employed in order to assist in reproduction. Human IVF typically involves superovulation of the ovaries induced by fertility drugs, and the subsequent harvesting of multiple eggs. Sperm from the male partner is then combined with the harvested eggs (sometimes using a procedure referred to as “ICSI”, wherein a single sperm is injected into the egg) to achieve fertilization. Thereafter, embryo culturing is conducted in a petri dish, as described above. The petri dish is placed in a large temperature and gas controlled incubator. The fertilization and culturing phase of the processes take place in a laboratory, typically under the direction of a PhD embryologist. Three to five days later, one or more embryos are transferred into the woman's uterus for implantation.

Non-human embryos are also grown in vitro for a variety of reasons. In fact, animal husbandry can be considered the precursor to embryo culturing techniques. Artificial insemination (Al) has been the predominant method used to increase the reproductive potential in livestock and other animals. In bovine (cow) artificial insemination, donor cows are superovulated and inseminated with semen from pedigree bulls. The resulting embryos are then recovered by flushing the uterine cavity for transfer to one or more surrogates. A large commercial industry of embryo transfer (ET) based on this process has been active since the 1950s. This industry, however, has seen little improvement in embryo development technology for nearly four decades.

Today, utilizing the standard in vitro fertilization technique of embryo culturing in a petri dish (as described above), oocytes (eggs) from the ovaries of high-grade donors can be combined with sperm from other high grade donors resulting in increased reproductive efficiency of genetically superior breeds. In fact, once an animal with the desired characteristics has been obtained, its DNA can be used in nuclear transfer (i.e., cloning) and subsequent embryo culturing, with the resulting embryos distributed around the world. Once again, however, the culturing phase of these processes requires highly skilled personnel and expensive, complex laboratory equipment. In spite of these drawbacks, high grade frozen bovine embryos are now commercially available on a limited scale for prices as high as $500 or more per embryo. Embryo culturing techniques in conjunction with in vitro fertilization or cloning may also be used to develop high pedigree breeds of animals for the show and gaming industries, as well as to reproduce endangered species for preservation.

Embryos may also be cultured for transgenic drug production, stem cell production, and xenograph production. With respect to transgenic drug production, it has recently been shown that it is possible to genetically modify an embryo with genes from a different species. These transgenic animals may then be employed as biological factories to produce useful hormones or other biological compounds. A transgenic animal is created by introducing foreign genes into a host's fertilized egg. The donor gene then fuses with the host genes and becomes part of the host DNA. The transgenic, fertilized egg is then cultured in vitro utilizing the petri dish techniques described above until it is ready to be transferred to a surrogate mother. All of the subsequent cells produced during embryonic development will inherit the foreign gene, and therefore the gene will be present in all of the cells of the resulting adult organism. The initial offspring produced by this method are called founder animals. These founder animals may be selectively bred, sometimes cloned, to create a production herd.

In the case of bovine transgenic animals, for example, therapeutic proteins or other extremely valuable biomaterials can be purified from the milk of these animals. Other transgenic animals currently being produced include mice, rabbits, goats, sheep, and pigs. One barrier to the rapid commercialization of these transgenic technologies, however, is the current labor-intensive, in vitro methods used in the micromanipulation and embryo culturing processes.

As for xenograph production, it is a well-known and an unfortunate reality that the demand for human organs for transplantation is far greater than the supply. Xenotransplantation is the transplantation of living cells, tissue, or organs from a non-human species into a human patient. For this application to be successful in human populations, the source animals must be genetically engineered to mimic autologous tissues (i.e., identical to the patient's own), thereby avoiding the immuno-suppression responses inherent in allogenic (different than the patient's own cells) transplants. Various techniques are employed to manipulate the donor animal's genes within the egg. After the egg is fertilized it is cultured in vitro utilizing current petri dish techniques and transferred to a surrogate mother. The xenographs are then harvested from the source animal at the appropriate time and transplanted to the patient.

One of the most exciting and potentially valuable developments in the biotech industry is the use of embryonic stem cells. Embryonic stem cells are derived from embryos that are cultured to the blastocyst stage utilizing current petri dish techniques. After extended culturing, the stem cells are harvested from the inner cellular mass of the blastocyst. These undifferentiated cells have the ability to grow into any specific kind of cell, tissue, or organ, and these “universal” cells may be used for the treatment of many disease processes. The extraordinary promise of using embryonic human stem cells to treat a wide range of human diseases has stimulated intense academic research, however further development has been hampered, in part, by the expensive, complex and time-consuming procedures currently used for embryo culturing (i.e., a petri dish in an incubator).

It should be apparent that embryo culturing is an important aspect of a variety of medical procedures. Despite these advancements in the various technologies which utilize embryos grown in vitro, culturing techniques have changed little over the years. The embryo is placed into a petri dish containing a suitable fluid growth media, and the petri dish is then placed into an incubator. Typically, the media is changed every 24 hours, generally by skilled embryologists, technicians, or physicians who physically move the embryo to another petri dish having fresh growth media. When IVF was first developed about 20 years ago, the embryo was transferred back to the mother about 24 hours after fertilization. Since that time, the timing of the embryo's transfer back into the body has increased from 1 day after fertilization to about 3-5 days after fertilization. Furthermore, the more recent uses of embryos grown in vitro (e.g., stem cell production) may require that the embryos are cultured for several more days. This can be difficult with conventional petri dish culturing, however, particularly when the fluid media is changed daily (or even more frequently). In addition, recent studies have suggested that a media suitable for the initial stages of embryo development may not be ideal during subsequent stages of embryo development. Although an embryo may be manually moved from one petri dish to another, it is desirable to minimize any physical manipulation of a developing embryo.

As can be seen, currently available equipment and techniques have a number of shortcomings that can greatly reduce the ability of an embryo to develop properly in vitro. For example, procedures using petri dishes and similar equipment require physical manipulation of the embryo, and moving the embryo from one petri dish to another to change the media solution.

### SUMMARY OF THE INVENTION

One embodiment of the present invention comprises a self-contained embryo support assembly which includes:

(a) a well for housing an embryo and a fluid therein;

(b) a control system for regulating one or more conditions within the well; and

(c) an energy source;

wherein the energy source is configured for powering the control system without connecting the embryonic support assembly to an external power source. The control system may comprise, for example, a heater configured for regulating the temperature within the well. The control system may further include one or more processors (such as a CPU), and at least one temperature sensor in electrical communication with the processor, wherein the processor is configured for regulating the heater. For example, the processor may regulate the operation of the heater in accordance with one or more sets of instructions provided to the processor from a memory. Such instructions may compare the sensed temperature to a set point, and thereafter adjust the operation of the heater in a manner intended to change the temperature of the well until it matches the set point (e.g., feedback control). The control system may also comprises at least one sensor chosen from the group consisting of a pH sensor, an oxygen sensor, a carbon dioxide sensor, an urea sensor, an ammonia sensor, a nitrogen sensor, a calcium ion sensor, a sodium ion sensor, a nitrate sensor, a phosphate sensor and an osmolarity sensor. The control system may cause the conditions within the well to be adjusted based upon a sensed property indicated by one or more of these sensors.

The embryonic support assembly may further include a fluid supply system configured for delivering fluid media to the well, and this fluid supply system may comprise a fluid reservoir in fluid communication with the well. The fluid reservoir may be pressurizable so that the fluid reservoir may be filled with a fluid media under pressure for delivery to the well. Alternatively, a pump may be used to deliver fluid media from the reservoir to the well. A valve for controlling the delivery of fluid media from the fluid reservoir to the well may also be included, and the pump and/or valve may be operated in accordance with one or more signals from the control system. A waste fluid reservoir in fluid communication with the well may also be included.

As mentioned above, the control system of the embryonic support assembly may be configured for regulating one or more conditions within the well according to a predetermined set of instructions (e.g., one or more sets of computer-readable instructions or algorithms stored in a memory which is in communication with the processor). The predetermined set of instructions may even be replaced or altered in response to a signal received from another device (such as an electronic signal received from an external computing device such as a computer which is in communication with the embryonic support assembly). The signal may even be received from a remote computing device over, for example, the Internet or other communications network

The embryonic support assembly may further include an identification means for identifying the embryonic support assembly or an embryo located therein, and this identification means may comprise, for example: printed indicia, memory (e.g., the memory described above), an etched indicia, an engraved indicia, a barcode, or even radioactive tagging of the assembly or the embryo itself. The embryonic support assembly may further be configured for periodically acquiring data concerning one or more conditions within the well and storing such data in the memory and/or transmitting such data to an external device.

The well of the support assembly may be formed by at least one of micromachining, microembossing and micromolding of a substrate. In fact, the assembly may comprise a MEMS device. In addition, the well may be configured such that an embryo located therein will tend to remain at a predetermined location within the well (e.g., at or near the bottom of the well due to gravity). For example, the well may have a tapered configuration such that an embryo located therein will be directed to the predetermined location within the well.

Another embodiment of the present invention provides a method for shipping a metabolically active embryo, comprising the steps of:

(a) providing a self-contained embryonic support assembly having a well housing an embryo and a fluid therein, and a control system for regulating one or more conditions within the well during shipment; and

(b) causing the embryonic support assembly to be transported from one location to another.

Yet another embodiment of the present invention comprises an embryo support assembly, comprising:

(a) a well for housing an embryo and a fluid therein; and

(b) a plurality of stations, each of which is configured for communication with the well;

wherein each of the plurality of stations are configured such that they may be selectively brought into communication with the well. At least one of the well and the plurality of stations may be selectively moveable with respect to one another such that each of the plurality of stations may be selectively brought into communication with the well. At least one of the plurality of stations may comprise a fluid reservoir for supplying fluid media to the well, while another station may comprise a passageway through which an embryo can be inserted into or removed from the well when the passageway is in communication with the well. Another station may optionally comprise a fertilization passageway sized and configured to allow the insertion of a fertilization device for injecting a sperm into an egg located within the well when the fertilization passageway is in communication with the well, and/or a visualization station.

Another embodiment of the present invention provides an embryo support assembly, comprising:

(a) at least two wells for housing an embryo and a fluid therein, the wells arranged vertically above one another; and

(b) at least one passageway which provides communication between the wells;

wherein the embryo support assembly is configured such that an embryo located within the uppermost well will travel downwardly through the at least one passageway into the second well under the action of gravity. This embryo support assembly may be configured for selectively controlling the movement of an embryo from one well to the next.

An embryo support system is also provided in accordance with another embodiment of the present invention, and this system comprises:

(a) at least one embryo support assembly having a well for housing an embryo and a fluid therein;

(b) a cartridge configured for removably receiving the at least one embryo support assembly;

(c) a base assembly configured for removably receiving the cartridge; and

(d) a control system for regulating one or more conditions within the well.

The control system may be provided in the embryo support assembly, and the cartridge may be configured for removably receiving a plurality of embryo support assemblies. For example, the cartridge may have a plurality of chambers, each of which is configured for removably receiving one of the embryo support assemblies. In addition, the chambers may be arranged in a circular, linear or rectilinear array.

Yet another embodiment of the present invention provides an embryo support device, comprising:

(a) at least one embryo support assembly having a well for housing an embryo and a fluid therein;

(b) a cartridge configured for removably receiving the at least one embryo support assembly.

A still further embodiment of the present invention is an embryo support device, comprising:

a. a plurality of wells, each configured for housing an embryo and a fluid therein; and

(b) a fluid supply system for each of the wells, each of the fluid supply systems comprising a plurality of fluid reservoirs in fluid communication with the well associated with the fluid supply system.

A control system for regulating one or more conditions within the wells may also be included, along with a plurality of imaging devices wherein each of the imaging devices is configured for acquiring image data of an embryo located within one of the wells. Similarly, a plurality of waste reservoirs, each of the waste reservoirs configured in fluid communication with one of the wells, may also be included. The embryo support device may be substantially disc-shaped, and the wells may be arranged in a substantially circular array.

### BRIEF DESCRIPTION OF THE DRAWINGS

While the specification concludes with claims particularly pointing out and distinctly claiming the present invention, it is believed the same will be better understood from the following description, taken in conjunction with the accompanying drawings in which:

FIG. 1 is a perspective view of one embodiment of an embryo support assembly according to one embodiment of of the present invention;

FIG. 2 is a schematic illustration, in transparent form, of the apparatus of FIG. 1;

FIG. 3 is a cross-sectional view of a portion of the apparatus of FIG. 2;

FIG. 4 is a cross-sectional schematic view of an alternative embodiment of an embryo support assembly according to one embodiment of the present invention;

FIG. 5 is a perspective view of the apparatus of FIG. 4;

FIG. 6 is a cross-sectional view of the apparatus of FIG. 2;

FIG. 7 is a cross-sectional view of a portion of the apparatus of FIG. 2;

FIG. 8 is another cross-sectional view of a portion of the apparatus of FIG. 2;

FIG. 9 is a cross-sectional, schematic view of the apparatus of FIG. 2;

FIG. 10 is a schematic view of the electronic components of the apparatus of FIG. 2;

FIG. 11 is a schematic view of a control system according to an embodiment of the present invention;

FIG. 12 is a perspective view of one embodiment of a cartridge according to one embodiment of the present invention;

FIG. 13 is a perspective, cut-away view of the cartridge of FIG. 12;

FIG. 14 is a perspective view of another embodiment of a cartridge according to one embodiment of the present invention;

FIG. 15 is a perspective view of another embodiment of a cartridge according to one embodiment of the present invention;

FIG. 16 is a perspective view of another embodiment of a cartridge according to one embodiment of the present invention;

FIG. 17 is a schematic illustration of one embodiment of a base assembly according to one embodiment of the present invention;

FIG. 18 is a perspective view of one embodiment of a base assembly according to one embodiment of the present invention;

FIGS. 19 through 23 are schematic illustrations of alternative embodiments for the embryo well and fluid reservoir components of an embryo support assembly according to one embodiment of the present invention.

FIG. 24 is a schematic illustration of an embryo well having a piezoelectric element therein;

FIG. 25 is a perspective view of an alternative embodiment of an embryo support assembly according to one embodiment of the present invention;

FIG. 26 is a top plan view of the embryo support assembly of FIG. 25;

FIG. 27 is a partially exploded view of the embryo support assembly of FIG. 25;

FIG. 28 is a cross-sectional view of the embryo support assembly of FIG. 25;

FIG. 29 is an exploded view of the embryo support assembly of FIG. 25;

FIG. 30 is a perspective, cross-sectional view of the embryo support assembly of FIG. 25; and

FIG. 31 is a schematic view of the embryo support assembly of FIG. 25.

### DETAILED DESCRIPTION OF THE PREFERRED EMBODIMENTS

One embodiment of the present invention provides a self-contained embryo support assembly that automatically maintains the appropriate environmental and/or metabolic conditions necessary to maximize the viability of an embryo contained therein. In fact, one embodiment essentially comprises an embryology “lab-on-a-chip” that allows embryo culturing to take place in a non-laboratory, production environment. The embryo support assembly may be reusable, or may even be manufactured for a single use, particularly since the fabrication techniques described herein may be used to inexpensively produce embryo support assemblies by well-known techniques.

The embryo support assembly may provide a closed, battery powered, self-contained system with internal environmental and/or metabolic controls. The user may simply place a fertilized oocyte into the embryo support assembly and the assembly will do the rest. For example, sterility, non-toxicity, temperature, and pH may be automatically maintained in the closed environment provided by the embryo support assembly. The conditions of the outside environment may have no effect on the conditions inside the assembly.

The embryo support assembly according to one embodiment of the present invention may also provide for growing an embryo in two or more different types of growth media. Currently, a highly-skilled technician must remove a petri dish containing an embryo from an environmentally-controlled incubation chamber and physically transfer the embryo to an already prepared second petri dish having different growth media therein. Such a process creates undo stress on the embryo and may limit its viability. The embryo support assembly according to one embodiment of the present invention may include several chambers (or reservoirs) preloaded with the necessary growth media. The media may be automatically regulated and its delivery to the embryo sequenced based on the requirements of the developing embryo. No user intervention may be required.

An embodiment of the embryo growth assembly of the present invention may also include an integrated visualization system for periodically or continuously monitoring and/or documenting the embryo growth process. Users can view the embryo in real time or use stop-action cinematography to compress long periods of low activity. The user may therefore be able to assess the health of the embryo by reviewing a video of its active development. Built-in sensors may also be included in order to measure and store the various conditions inside the development chamber (i.e., the well described below).

The self-contained, self-powered feature of one embodiment of the present invention also allows for the shipment (i.e., transportation from one location to another) anywhere in the world without freezing or external support systems. The embryo support assembly of the present invention may also be a MicroElectroMechanical System device (a “MEMS device”) which includes microfluidic controls. MEMS devices generally include mechanical microstructures, microsensors, microactuators, and electronics integrated onto a single chip.

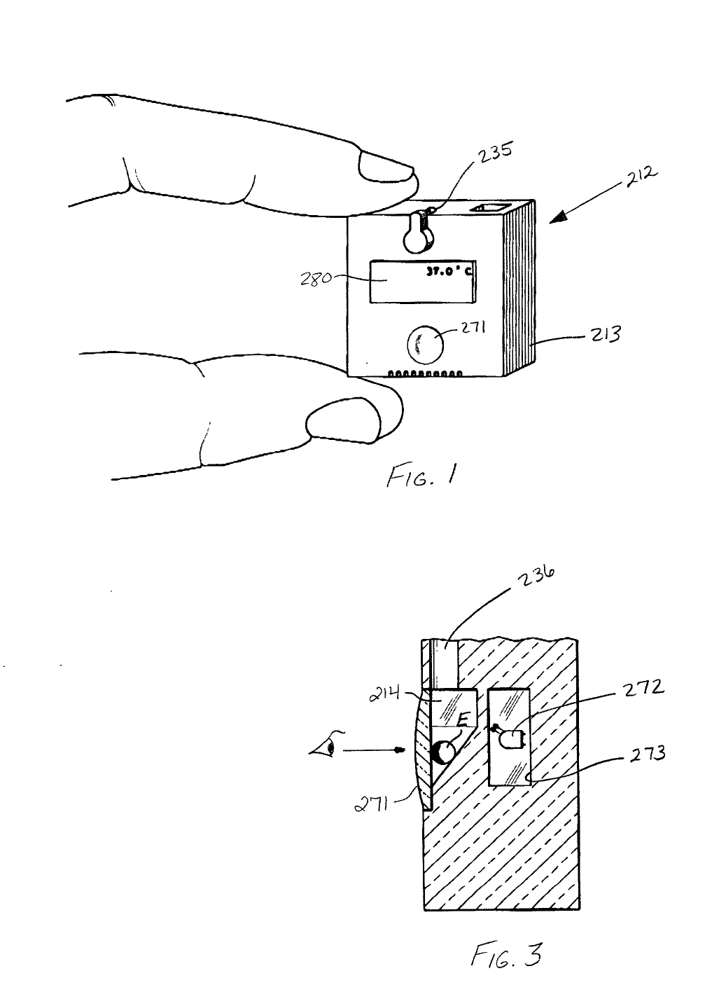

Referring now to the drawing figures, wherein like numerals indicate similar elements throughout the views, FIG. 1 depicts a self-contained embryo support assembly according to one embodiment of the present invention. This support assembly may be used in the fertilization of an ovum with sperm, and/or the development of an embryo for a period of time after fertilization. As would be contemplated and understood by those skilled in the art, the present invention can be adapted for use with the embryos of any animal, including human or other mammalian ovum and sperm. It should be pointed out that the present invention is in no way limited to use with conventional in vitro fertilization techniques. Rather, the present invention can be used for any type of embryo, including those produced, for example, by cloning techniques.

The self-contained embryo support assembly 212 depicted in FIG. 1 is self-contained in that it includes its own power supply, and is configured for maintaining the viability of a one or more ovums or one or more live embryos (particularly, unfrozen embryos) therein without the need for an external source of power. In the embodiment shown, no other external inputs are necessary in order to maintain an ovum or a live embryo therein, such as external fluid inputs and the like. Thus, once an ovum or embryo is located within embryo support assembly 212, the support assembly may remain unattended, with the ovum or embryo remaining viable for later fertilization, implantation or cryogenic preservation.

As also seen in FIG. 1, the embryo support assembly 212 may be manufactured such that the assembly is sized so as to be handheld. For example, assembly 212 may be sized such that its width (or diameter) in any direction is less than about 4 inches, or even less than about 2 inches, thereby allowing assembly 212 to be easily held in one's hand and even operated or otherwise manipulated while being held in one's hand. Furthermore, the small size of assembly 212, as well as its self-contained nature, allows embryo support assembly 212 to be shipped (i.e., transported from one location to another) with a viable ovum or embryo (particularly an unfrozen, developing embryo) located within the support assembly, without the need for providing an external power supply or other external inputs during shipment. It should be pointed out that, although one embodiment of the embryo support assembly of the present invention is sized so as to be handheld, other embodiments of the present invention are not necessarily limited to handheld, or unconventionally sized, embryo culturing devices. Thus, certain embodiments of the present invention are in no way limited to handheld or otherwise unconventionally sized devices.

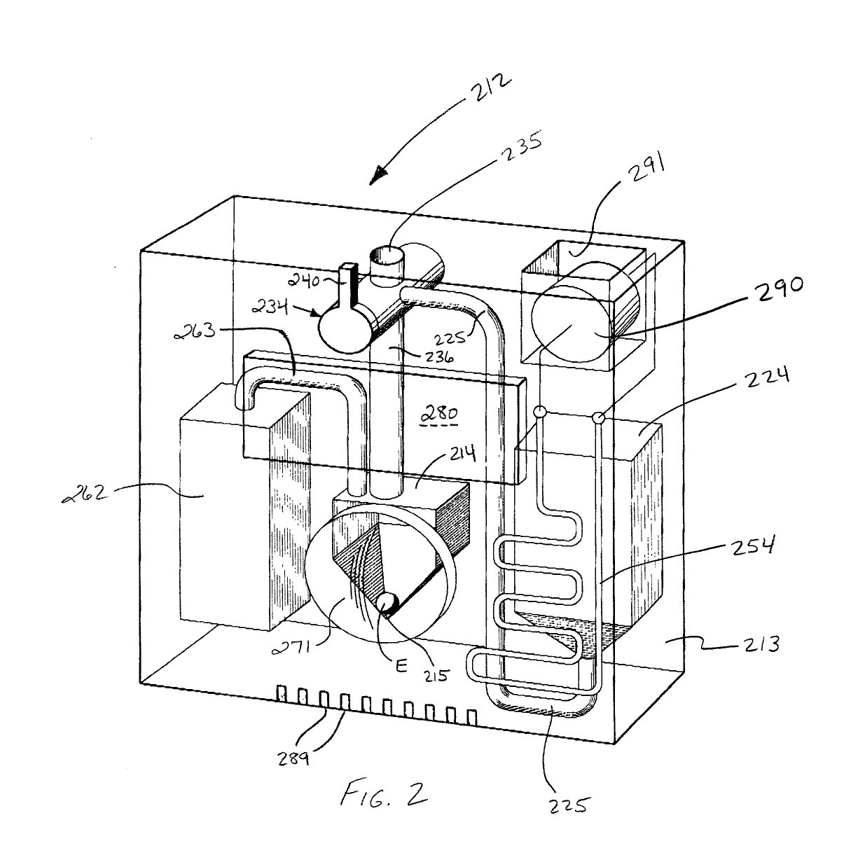

In order to provide a handheld device, one embodiment of the embryo support assembly of the present invention can comprise a microfabricated device, thus providing a miniaturized, self-contained embryo support assembly. In fact, support assembly 212 can comprise a microchip having a micromachined well 214 for the ovum or embryo (see FIG. 2), as well as various fluid media reservoirs and fluid channels through which fluid media may pass. Microfluidic controls may also be provided on the microchip (such as one or more various types of microfluidic valves and/or pumps), as well as “on chip” electronics (provided, for example, in the form of one or more integrated circuits fabricated directly on the microchip).

It should be pointed out that, since the various devices and assemblies described herein may not only be used for maintaining a growing embryo in a viable condition, but also for fertilizing an oocyte with sperm, well 914 may also be characterized as a fertilization or culturing well to reflect the fact that its use is not limited to growing or maintaining embryos therein. Therefore, the present invention is deemed to encompass the use of such alternative terminology and applications.

The embryo support assembly according to one embodiment of the present invention may comprise a microchip fabricated from a single, solid substrate, such as silicon, glass, quartz, plastic or metal. The embryo well(s), as well as the various channels and reservoirs (as further described below), for example, may be fabricated directly on this substrate. These wells, channels and reservoirs (as well as other components of the embryo support assembly) can be micromachined, microembossed and/or micromolded in the substrate by any of a variety of methods well known to those skilled in the art, including film deposition processses such as spin coating and chemical vapor deposition, laser fabrication, LIGA (a type of micromolding), photolithographic techniques, or etching (wet chemical or plasma). At the same time, the various electronic components (further described herein) may be integrally formed on the substrate (“on chip” electronics), such as in the form of one or more integrated circuits, using these same methods.

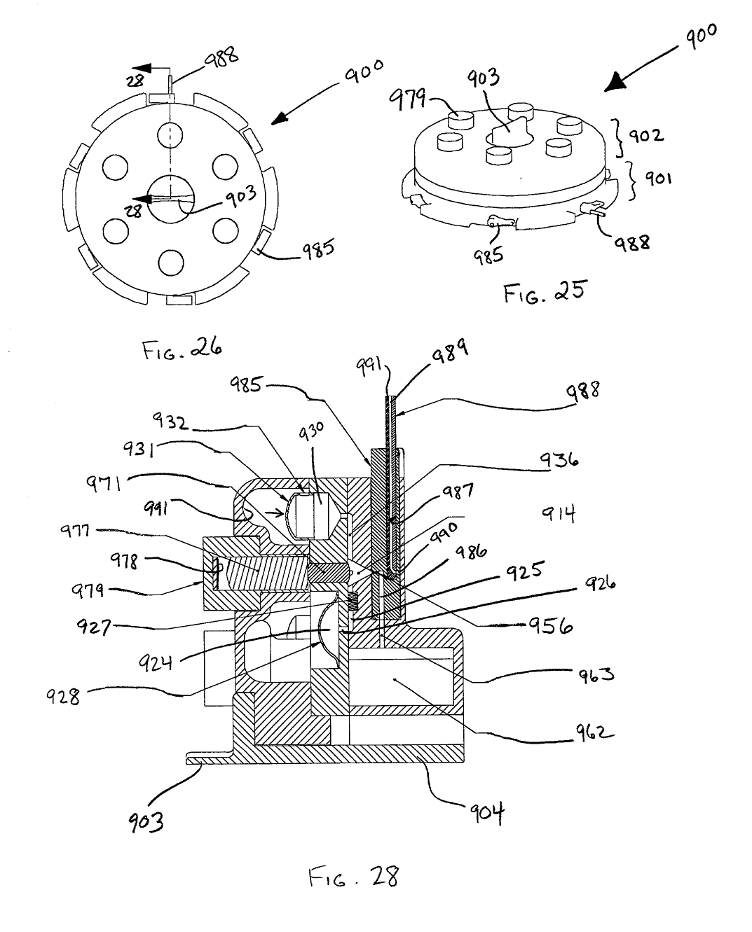

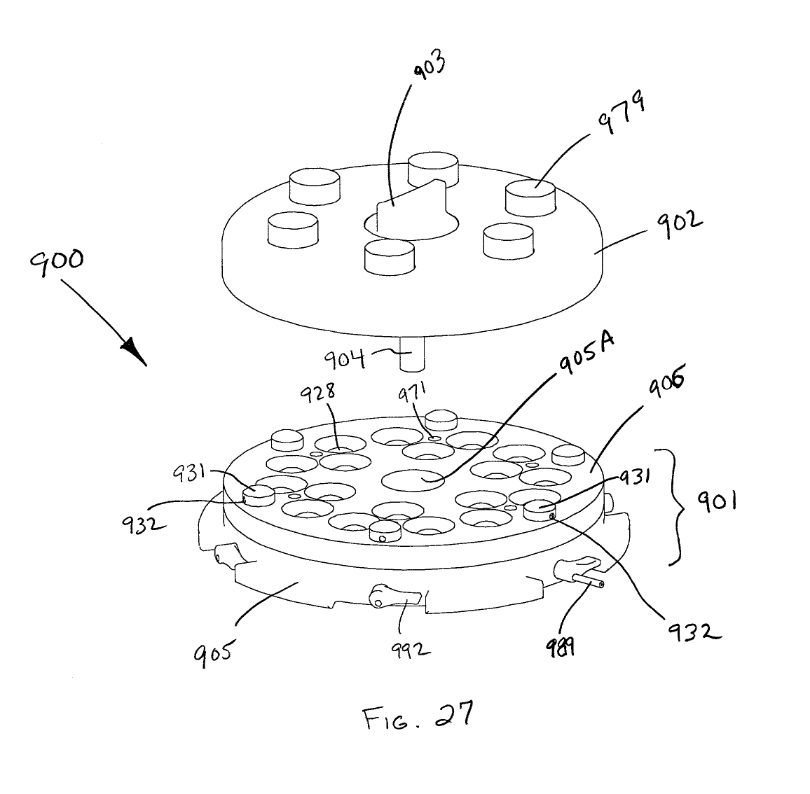

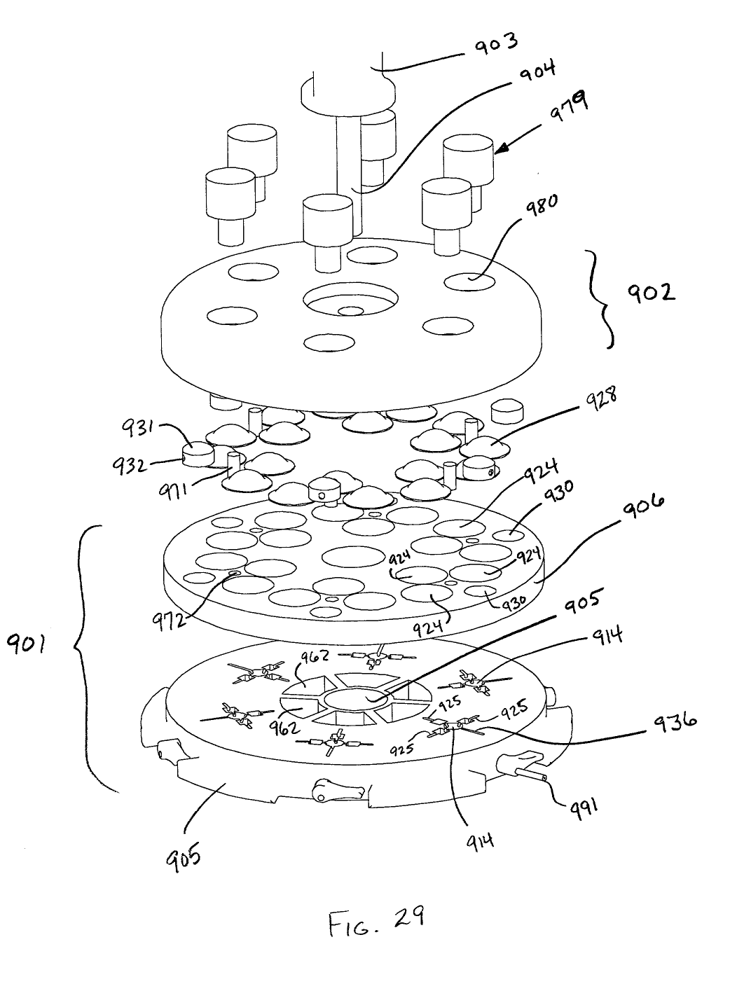

It should also be pointed out that, while one embodiment of the present invention provides a handheld embryo support assembly fabricated on a single substrate, it is also contemplated that multiple substrate layers may also be employed. For example, the embryo well may be micromachined (e.g., by etching), microembossed and/or micromolded on a substrate, other components (e.g., the various electronic components or a visualation window or lens) may be provided on one or more additional substrates which are bonded or otherwise attached to the substrate in which the embryo well is formed (for example, the embodiment shown in FIGS. 25-30).

As described above, one embodiment of the embryo support assembly of the present invention comprises a substrate in which at least one of the embryo well, fluid reservoirs, and fluid channels are micromachined (such as by photolithography or etching) or micromolded (e.g., using well-known LIGA techniques) in a substrate. FIG. 2 is a schematic illustration of embryo support assembly 212 fabricated on a substrate 213. It should be pointed out that substrate 213 is depicted as being transparent for purposes of clarity, and substrate 213 need not be transparent. In addition, various electronic components and circuitry may also be provided (e.g., a CPU, memory, etc.) on embryo support assembly 212, however these components (except for a power supply and a heater) are not depicted in FIG. 2 for purposes of clarity.

As seen in FIG. 2, embryo support assembly 212 may include a well (or tank) 214 which is configured for housing an embryo E therein. Alternatively, well 214 may be used to house an ovum (i.e., an unfertilized egg) therein. In fact, as further detailed herein, embryo support assembly 212 may be employed for the fertilization of an ovum located within well 214. Well 214 is sized to hold a sufficient amount of fluid to allow for the fertilization of an ovum by sperm and/or to allow for the development of an embryo E housed therein.

Well 214 also may be configured such that an ovum or embryo housed therein will be located in a predetermined region of well 214. By way of example, well 214 may be configured such that gravity will urge embryo E into the predetermined location, such as the lowermost central portion of well 214. In the embodiment of FIG. 2, well 214 includes one or more tapered sidewalls which converge downwardly towards the lowermost portion of well 214. In the particular embodiment of FIG. 2, the sidewalls of well 214 taper downwardly towards the center of well 214 and terminate at a base point 215 of well 214. In this manner, an embryo E located within well 214 will, under the force of gravity, naturally tend to be located adjacent to base point 215 of well 214 (as shown in FIG. 2). As will be apparent, such a configuration not only facilitates the visualization of an ovum or embryo located within well 214, but also facilitates extraction of the embryo from the embryo support assembly and/or facilitates the fertilization of an ovum positioned within well 214. It should be kept in mind, however, that the particular configuration for embryo well 214 shown in FIG. 2 is merely one possible embodiment, since a variety of other configurations may be employed.

Embryo support assembly 212 also may include a passageway 236 which is in fluid communication with well 214, and which terminates in a port 235 through which access to well 214 may be obtained. In this manner, passageway 236 may be used to insert an embryo into, or remove an embryo from well 214. In addition, as further described herein, an unfertilized egg may be placed into well 214 through passageway 236, and thereafter fertilized directly within well 214 (with sperm introduced into well 214 through passageway 236). It will be understood, however, that additional passageways and associated ports may also be provided on embryo support assembly 212. For example, a separate passageway and associated port may be provided for purposes of embryo removal (i.e., one port for embryo or ovum insertion, and another port for embryo removal).

A valve 234 may also be provided in order to selectively open and close passageway 236. As further described herein, valve 234 may serve additional purposes such as providing fluid communication between well 214 and a fluid media reservoir 224, as well as providing fluid communication between reservoir 224 and the ambient (e.g., for charging reservoir 224 with a fluid media).

As more fully described herein, embryo support assembly 212 may be utilized for maintaining an ovum or an embryo E within well 214 in a suitable fluid media. During such time, it may be desirable to monitor (e.g., monitor, examine, record, and/or view) fertilization of the ovum by sperm and/or the development of the embryo without physically manipulating the ovum, sperm and/or embryo E. For example, an embryo E maintained within well 214 will continue to divide during the period of time it resides within well 214, and it may be desirable to visually monitor the development of embryo E while it is maintained within well 214. Thus, embryo support assembly 212 may also be configured to provide for visualization of Embryo E.

A variety of devices and systems may be employed to provide for visualization of the an ovum or embryo within well 214. For example, in the embodiment of FIG. 2, a transparent window, such as in the form of a lens 271, may be provided adjacent well 214 in order to provide optical communication between the interior of well 214 and the exterior of assembly 212. In this manner, embryo E may be visualized through lens 271 (e.g., by the use of a viewing device such as a microscope or other suitable device which provides magnified viewing). Of course any number of windows may be provided, and it is not necessary that such windows comprise a lens (or lens elements). As an alternative to providing a separate window 271 which provides optical communication between the ambient and the interior of well 214, it is also contemplated that substrate 213 from which the embryo support assembly 212 is manufactured can comprise a transparent material. In this manner, visualization of the embryo may be obtained directly through substrate 213.

The window, lens or other transparent region providing optical communication between the interior of well 214 and the exterior of assembly 212 may also comprise at least a portion of a wall of embryo well 214. Thus, in the embodiment of FIGS. 2 and 3, lens 271 provides the entire front wall of well 214, as best seen in the cross-sectional view of FIG. 3. During fabrication, for example, after well 214 has been micromachined into substrate 213, a transparent cover plate, such as a glass or plastic lens 271, may be sealed to substrate 213 over top of embryo well 214, such that the cover plate (i.e., lens 271) becomes a wall (or a portion of a wall) of embryo well 214 (as shown in FIG. 3).

In order to provide for adequate visualization of embryo E within well 214, it may also be desirable to illuminate the interior of well 214. When substrate 213 is transparent, an external light source may simply be placed next to apparatus 212 for illumination purposes. Alternatively, light from an external light source may be directed through lens 271, or another transparent window or lens providing optical communication between the interior of well 214 and the ambient. For example, the rear wall of well 214 may be provided, at least in part, by another lens or a simple transparent window. An ovum or embryo located within well 214 may then be backlit simply by directing light from an external light source through the transparent portion of the rear wall of well 214.

While an external light source may be used to illuminate an ovum or an embryo E, the embodiment of FIG. 3 includes a light source 272 located within embryo support assembly 212. It should be noted that light source 272 is not shown in FIG. 3 for purposes of clarity. Light source 272 can comprise any of a variety of devices suitable for illuminating embryo E within well 214. For example, light source 272 can comprise one or more LED's or laser diodes located within a chamber 273 formed within substrate 212. The region between chamber 273 and well 214 may also be translucent or transparent so that light from light source 272 may enter well 214 for illumination purposes. Light source 272 (e.g., LED's or laser diodes) may even be formed directly on the substrate by well-known techniques. Light source 272 may also be configured to provide polarized light (particularly circular polarized light) in order to improve image contrast.

While the embodiment of FIG. 3 provides for visualization of embryo E through window or lens 271, other embodiments of the present invention may include a visualization assembly configured for visualization of an embryo within well 214. By way of example, it may be desirable to perform sequential and/or continuous observation of an ovum or an embryo (e.g., to monitor the progress of fertilization and/or embryo development). In fact, it is contemplated that such visualization (i.e., observation) will assist in selecting those embryos which are most likely to result in a successful pregnancy upon implantation. Although visualization using a manual, external magnifying device can provide for sequential imaging (e.g., periodically observing the ovum or embryo through lens 271), a visualization assembly (i.e., an imaging device) may be provided.

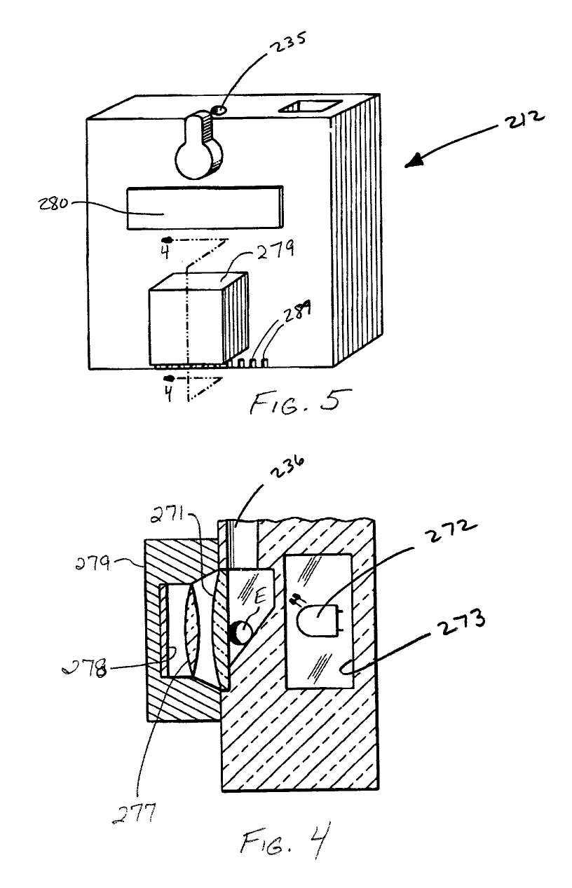

Although an imaging device may be integrally provided on embryo support assembly 212, one embodiment of the present invention provides a removable imaging device. As best seen in FIGS. 4 and 5, the imaging device may comprise a charge coupled device (or CCD) 278. Alternatively, a CMOS image sensor array may be used. CCD and CMOS imagers generally comprise sensor arrays, wherein each of the sensors is light responsive. The CCD or CMOS imager acquires image data based upon the amount of light reaching the individual sensor elements of the array. The array may be housed in a circuit package which is electrically connected (e.g., by a cable or other current conducting medium) to supporting electronics for processing and storage of image data. In one embodiment, CCD 278 may be connected to to a computer or other external electronic device for image processing, storage and visualization. For example, an external computer (such as a general purpose computer or a PC) may receive image data from CCD 278, process such data in order to generate a digitized image, store the digitized image (e.g., as a data file in memory), display the digitized image on a computer monitor, and even print the image onto paper or other suitable substrate. It should also be pointed out that any of a variety of imaging devices (i.e., one or more light responsive sensors) may be used in place of a CCD or CMOS device.

As further described herein, apparatus 212 according to one embodiment of the present invention may also integrally include electronic components and circuitry, including, for example, one or more processors, such as a CPU, as well as memory (e.g., both RAM and ROM). CCD 278 (or other imaging device) may be in electrical communication with the electronic components of apparatus 212 such that the image data generated by CCD 278 may be processed by apparatus 212 itself and even stored (e.g., as one or more image data files in memory) in apparatus 212. Embryo support assembly 212 may even be configured such that a CPU (or other processing device therein) acquires periodic image data from CCD 278 and stores such image data (or other data such as a digitized image derived from the imaging data provided by CCD 278). In this manner, embryo support assembly 212 may periodically acquire, store and even analyze image data provided by CCD 278.

Although CCD 278 may be integrally provided on substrate 213, the embodiment shown in FIGS. 4 and 5 includes an imaging housing 279 which accomodates CCD 278. Housing 279 is configured for attachment to the front of embryo support assembly 212, as shown schematically in FIGS. 4 and 5. Suitable alignment and attachment features may be provided on housing 279 and embryo support assembly 212, such that imaging housing 279 may be alignably attached to support assembly 212 (i.e., so that CCD 278 may acquire image data indicative of the interior of well 214, particularly the region of well 214 whereat an ovum or embryo is located). One or more additional lens elements 277 may also be provided within imaging housing 279 such that when housing 279 is attached to embryo support assembly 212, lens element(s) 277 will be located between CCD 278 and embryo well 214. In this manner, lens element(s) 277 can be employed to suitably magnify and/or focus an image of an ovum or embryo within well 214 on CCD 278. Electrical connections may also be provided between imaging housing 279 and embryo support assembly 212, such that image data from CCD 278 may be transmitted to the electronic circuitry of embryo support assembly 212.

As further described herein, support assembly 212 may include one or more electrical contacts suitable for providing electrical communication between embryo support assembly 212 and one or more external electronic devices (such as a computer or a computer network). In this manner, the signal representative of an image of embryo E provided by CCD 278 may be transmitted from embryo support assembly 212 to a computer or other external device for processing, storage, analysis and/or visualization. Of course, as mentioned above, one or more of these steps may be performed within embryo support assembly 212. By way of example, embryo support assembly 212 may acquire image data from CCD 278, process such data to provide digitized images of an ovum or embryo, store such digitized images in memory, and thereafter transmit the stored images to an external device such as a computer for purposes of storage, analysis and/or visualization (e.g., displaying the digitized images on a computer monitor).

In order to provide a suitable environment for an ovum or an embryo, support assembly 212 may include a control system for regulating one or more conditions within well 214. For example, embryo support assembly 212 may be configured to maintain an ovum or an embryo within a fluid media in well 214 at a temperature suitable for maintaining ovum or embryo viability. During the time that an ovum or an embryo E is maintained within well 214, it will often be necessary to maintain the fluid media within well 214 at a temperature elevated above the ambient temperature. For example, the optimum fluid media temperature for maintenance of a human embryo is currently believed to be between about 97 and about 99° F. Therefore, the embodiment of embryo support assembly 212 depicted in FIG. 2 includes a control system which comprises at least one heater 254 in order to regulate the temperature within well 214.

In the exemplary embodiment of FIG. 2, heater 254 comprises a resistive heating element, wherein heat is emitted therefrom as current is passed therethrough. In addition, when heater 254 comprises a resistive heating element, the amount of heat emitted therefrom is proportional to the current passing therethrough. In this manner, by controlling the current to heater 254 (e.g., by using a feedback or feedforward control scheme) the temperature within well 214 may be maintained at the desired temperature. Resistive heating element 254 may be provided in a variety of configurations, and that shown in FIG. 2 is merely exemplary of one contemplated configuration. In addition, resistive heating element 254 may be readily formed during the fabrication process by any of a variety of well-known microfabrication techniques. It is further contemplated that other types of heaters may be provided (such as one or more infrared diodes), as well as multiple heaters.

Embryo support assembly 212 may also include an energy source configured for powering not only heater 254 but also other electronic components of embryo support assembly 212 (e.g., a CPU, CCD 278, or other type of imaging device). A variety of energy sources may be used, such as one or more solar cells or various types of power storage devices (e.g., one or more batteries). In the exemplary embodiment of FIG. 2, the energy source for embryo support assembly 212 comprises a battery 290 removably positioned within support assembly 212. A battery chamber 291 may also be formed within assembly 212 (e.g., within substrate 213) in order to accommodate battery 290 therein. In this manner, battery 290 may be replaced as needed (e.g., when the battery is exhausted). Of course the present invention also contemplates a power supply (such as a battery) which is integrally formed during the fabrication of embryo support assembly 212, or a battery which is otherwise not intended to be replaced (e.g., a rechargeable battery). It should also be pointed out that a suitable cover or other enclosure (not shown in the attached figures) may be provided in order to close or seal battery chamber 291 when battery 290 has been inserted therein.

As mentioned previously, and as further described in detail below, embryo support assembly 212 may also include a variety of additional electronics, such as various electronic components and circuitry provided in the form of one or more integrated circuits. Such electronics can comprise, for example, additional components of a control system for regulating the output of heater 254 based upon a sensed temperature within embryo well 214, as well as for regulating other conditions within well 214. For purposes of clarity, however, FIG. 2 merely depicts a direct electrical connection between resistive heating element 254 and the two poles of battery 290. While such a configuration would provide for a constant heat output from resistive heating element 254, other embodiments of embryo support assembly 212 may include additional electronics not shown in FIG. 2.

Some embodiments of the embryo support assembly according to the present invention further include one or more fluid media reservoirs configured for providing fluid media, such as embryo growth media, to well 214. In the embodiment of FIG. 2, for example, a fluid media reservoir 224 is provided. A fluid media suitable for in vitro fertilization of an ovum or embryo maintenance and/or development (embryo growth media) may be provided within reservoir 224, and may even differ from a fluid media initially provided within well 214.

For example, recently it has been determined that, for some embryos, it may be desirable to employ one type of fluid media for the first few days following fertilization, and a second, different type of fluid media thereafter. Thus, embryo support assembly 212 may be provided with the first type of fluid media (“Fluid Media I”) already within embryo well 214 even before an embryo is placed therein (i.e., pre-loaded with Fluid Media I). Once an embryo is placed within well 214, additional fluid media (Fluid Media I, or a different type of fluid media such as “Fluid Media II”) may be urged into embryo well 214 at a predetermined time or even at a time determined on the basis of the actual development of an embryo within well 214 and/or sensed conditions within well 214. Thus, fluid media reservoir 224 may be placed in fluid communication with embryo well 214 such that the fluid media contained within reservoir 224 may be urged into well 214. In the embodiment shown in FIG. 2, a fluid outlet is provided at the base of reservoir 224, and is in fluid communication with a fluid channel 225. At its opposite end, fluid channel 225 may be in fluid communication with embryo well 214 such that fluid may be urged from reservoir 224 into embryo well 214. Any type of fluid media suitable for embryo support/development known to those skilled in the art (or hereafter developed) may be used.

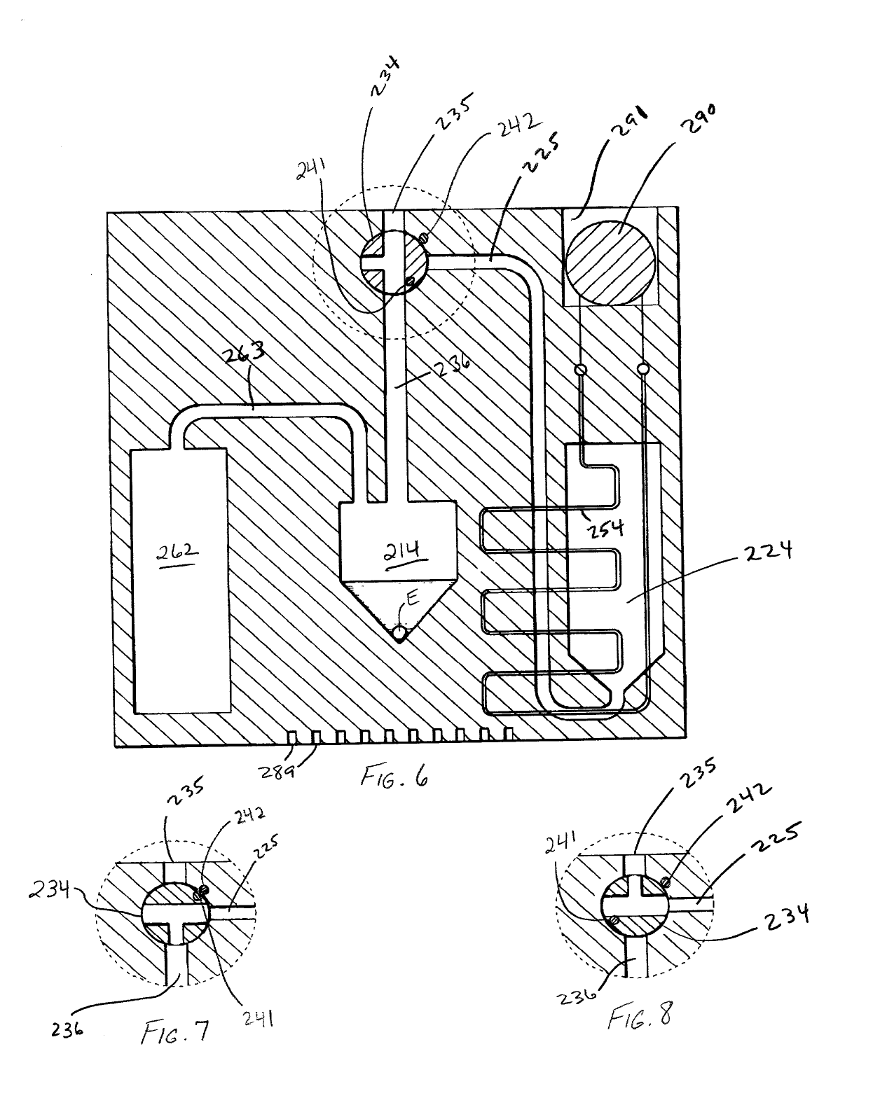

A distinct fluid channel may be provided between fluid media reservoir 224 and well 214 for delivering fluid from reservoir 224 to well 214. Alternatively, in the embodiment of FIG. 2, fluid channel 225 is in fluid communication with embryo well 214 through passageway 236, via valve 234. Valve 234 may be configured such that, depending upon the positioning of valve 234, passageway 236 is either in fluid communication with the external environment through port 235 (as shown in FIG. 6), or is in fluid communication with fluid channel 225 (as shown in FIG. 7). When valve 234 is positioned to provide fluid communication between passageway 236 and fluid channel 225 (e.g., by rotating valve 234 to the position shown in FIG. 7), passageway 236 will no longer be in communication with port 235, thus sealing embryo well 214 from the environment. At this position, fluid media urged from reservoir 224 will travel through fluid channel 225 into passageway 236, and ultimately into embryo well 214. In this manner, additional fluid media may be urged from reservoir 224 into embryo well 214.

Valve 234 may also be rotated to provide fluid communication between fluid channel 225 and the external environment through port 235 (as shown in FIG. 8). In this manner, reservoir 224 may be charged with fluid media through port 235. Although a variety of valve types may be employed, in the embodiment shown in FIGS. 6-9, valve 234 comprises a three-way valve (such as a three way stopcock valve).

Fluid media may be urged from reservoir 224 into embryo well 214 in a variety of manners. For example, as is well known to those skilled in the art, a variety of pumping devices may be microfabricated directly on the chip (i.e., substrate 213). Similarly, a variety of valves may also be microfabricated directly on the chip in order to provide for the controlled release of fluid media into embryo well 214. In fact, the control system of the embryo support assembly may be used to control the operation of valves and pumps which control the delivery of various fluids to well 214.

In the embodiment of FIG. 2, however, the fluid media within reservoir 224 is pressurized, such that when fluid communication is provided between reservoir 224 and embryo well 214, the pressurized fluid media will be urged into embryo well 214. In this configuration, additional pumps are generally not necessary in order to urge fluid from reservoir 224 into embryo well 214. Furthermore, since embryo support assembly 212 generally comprises micromachined fluid reservoirs, channels and passageways, fluid channel 225 will generally have a microscopic cross-sectional area. As such, fluid channel 225 (or one or more orifices therein) may be readily configured such that fluid mechanics will dictate a predetermined flow rate of fluid media from reservoir 224 into embryo well 214 based on the amount of fluid pressure within reservoir 224 and the configuration and size of fluid channel 225. In this manner, some embodiments of the present invention will not require control valves and the like in order to control the flow of fluid media from reservoir 224 into embryo well 214.

Other embodiments of the present invention may include one or more pumps and/or control valves in order to control the delivery of fluid media from reservoir 224 to embryo well 214. In fact, since the fluid channels will generally have a microscopic cross-sectional area, a pump alone may even be employed to deliver fluid media to well 214. For example, fluid channel 225 may comprise a “hydrophobic valve” well-known to those skilled in the art. Such a hydrophobic valve does not technically include an actual valve mechanism. Rather, fluid channel 225 may comprise a very small capillary coated with a hydrophobic material chosen to repel the fluid media housed within reservoir 224, and will therefore not allow the fluid media to flow therethrough unless the fluid media is of a sufficient pressure. In this manner, a micromachined pump (well-known to those skilled in the art), for example, may be provided along fluid channel 225 so that the fluid media from reservoir 224 may be selectively pressurized to a pressure sufficient to urge the fluid media through channel 225 having the hydrophobic coating therein. In addition, such hydrophobic valves (i.e., a suitably-coated fluid channel 225) also allow for the control of the fluid flow rate therethrough. By controlling the pressure delivered by the fluid media pump, the flow rate of the fluid media from reservoir 224 into well 214 may be precisely controlled, as desired.

Capillary electrophoresis may also be used to deliver fluid media from reservoir 224 to well 214. In such systems, an EMF field around a capillary (such as fluid channel 225) may be used to cause fluid flow through the capillary, without the need for a pump. Thus, the embryo support assembly according to one embodiment of the present invention may include a device for generating such an EMF field around one or more of the fluid media channels (such as fluid channel 225) in order to cause fluid flow therethrough (such as causing fluid media to be delivered from reservoir 224 to well 214). Such devices are completely solid-state, with no moving parts. Therefore, fabrication of the embryo support assembly may be simplified, and the use of pumps and/or valves may be avoided (particularly since the flow of fluid may be controlled simply by controlling the magnitude of the EMF field). Further alternative mechanisms for delivering fluid media from reservoir 224 to well 214 (or delivering fluid media through other channels of the embryo support assembly) may include one or more diaphragm pumps driven by heat, or various other devices known to those skilled in the art (particularly pumps and/or valves which may be produced using microfabrication techniques).

It should be pointed out that the dimensions of the fluid channels in the embryo support assembly shown in the accompanying figures have been greatly exaggerated for purposes of clarity. It should also be pointed out that well 214 may be of an extremely small size (smaller, in fact, than that indicated in the accompanying figures), particularly since the size of embryo E is greatly exaggerated in the figures.

As stated previously, in the embodiment of FIG. 2, embryo well 214 may be filled with fluid media whenever an ovum or embryo E is located therein. Therefore, as fluid media from reservoir 224 is added to embryo well 214, the same amount of fluid generally should be removed from embryo well 214 in order to avoid a pressure increase in well 214 (of course in some instances it may be desirable to vary the fluid pressure within well 214 as a function of, for example, embryo development). Thus, the embryo support assembly of FIG. 2 may include a fluid waste reservoir 262 in fluid communication with embryo well 214 through waste fluid channel 263. In this manner, as new fluid media is urged from reservoir 224 into embryo well 214, a portion of the fluid already in well 214 will be urged out of well 214 through waste fluid channel 263 into waste reservoir 262.

It should be noted that the configuration of the fluid media and waste reservoirs shown in the accompanying figures, as well as that of the various channels through which the fluid is urged into and out of embryo well 214, is merely exemplary. Thus, a variety of alternative configurations are contemplated and included within the scope of the present invention. For example, although waste fluid channel 263 is depicted in FIG. 2 as exiting the upper portion of well 214, it is also contemplated that waste fluid channel 263 may enter well 214 near the base thereof such that the fluid media will generally flow downwardly through embryo well 214. This downward flow will not only assist in insuring that embryo E is located in the lowermost portion of embryo well 214, but will also create a fluid flow across the surface of an ovum or embryo E. In addition, such a configuration further ensures that the majority of fluid leaving embryo well 214 through waste fluid channel 263 comprises “old” fluid media and not the “new” fluid media entering well 214 from reservoir 224.

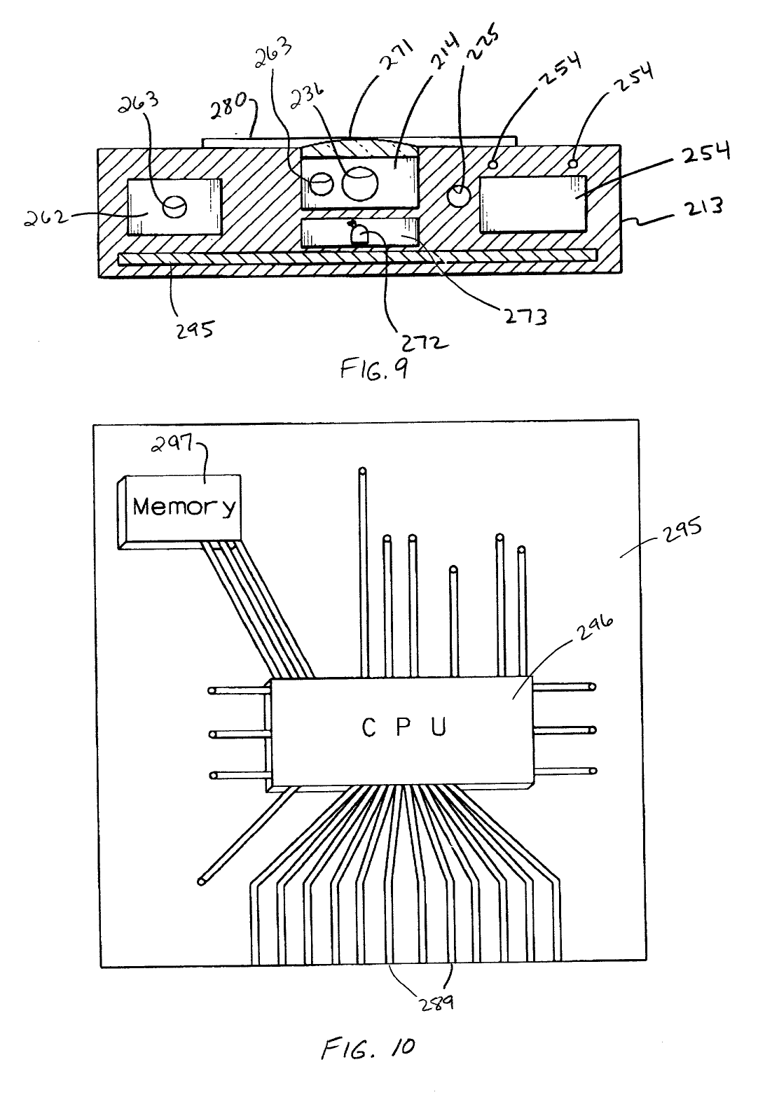

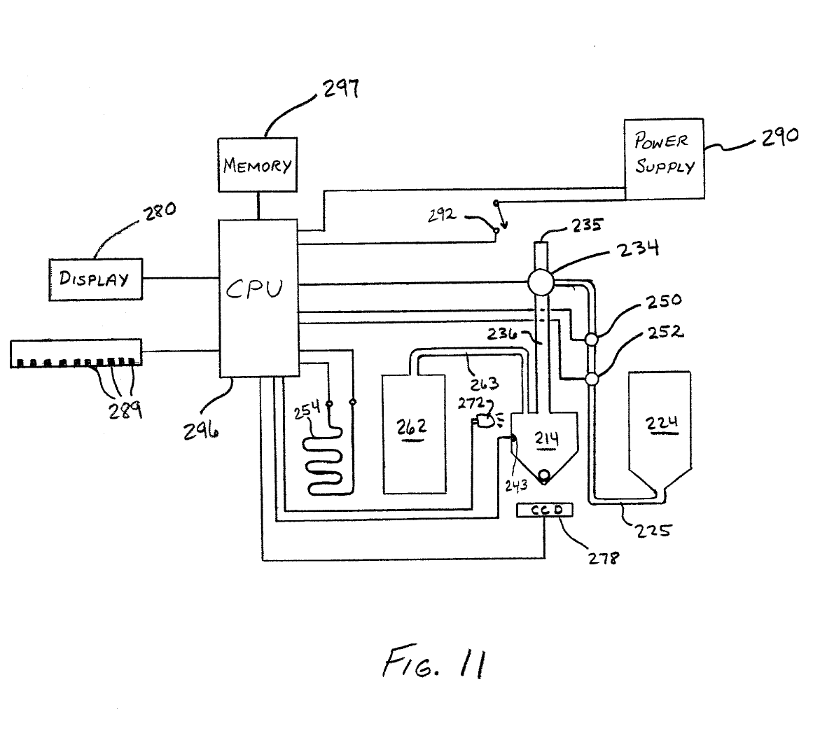

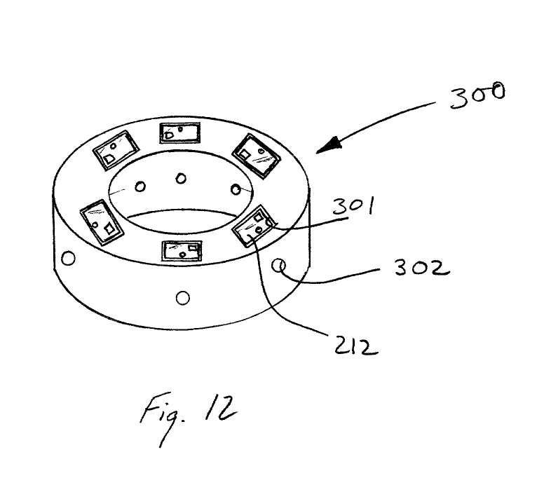

As mentioned above, assembly 212 may include additional electronic componentry, particularly in the form of one or more integrated circuits provided directly on substrate 213. Such electronics can be readily formed on substrate 213 by any of a variety of well-known fabrication techniques, and may be located in any of a variety of locations on the substrate. By way of example only, these electronics may be provided as one or more integrated circuits located in a discrete layer or plane in assembly 212. Thus, as shown in the schematic, cross-sectional view of FIG. 9, an integrated circuit (“IC”) 295 may be provided in a plane located behind the microfluidic components (i.e., well 214, reservoirs 224 and 262, and associated fluid channels). The electronic components of IC 295 may comprise any of a variety of elements, particularly additional components of the control system for assembly 212. As seen in the schematic view of FIG. 10, IC 295 may include a processor (e.g., CPU 296), memory 297, and various electrical leads which provide electrical communication between IC 295 and other components of assembly 212 (e.g., sensors, valves and/or pumps). Memory 297 may store any of a variety of types of information, including instructions to be executed by CPU 296 (e.g., in the form of one or more instruction sets), acquired data and images, and even identification information for identifying the assembly or the embryo contained therein. Of course other identifiers may be used for identifying assembly 212 or an embryo therein, such as printed indicia printed indicia, an etched indicia, an engraved indicia, or a barcode on the assembly itself. The assembly or even the embryo housed therein, may also be radioactively tagged for identification purposes.

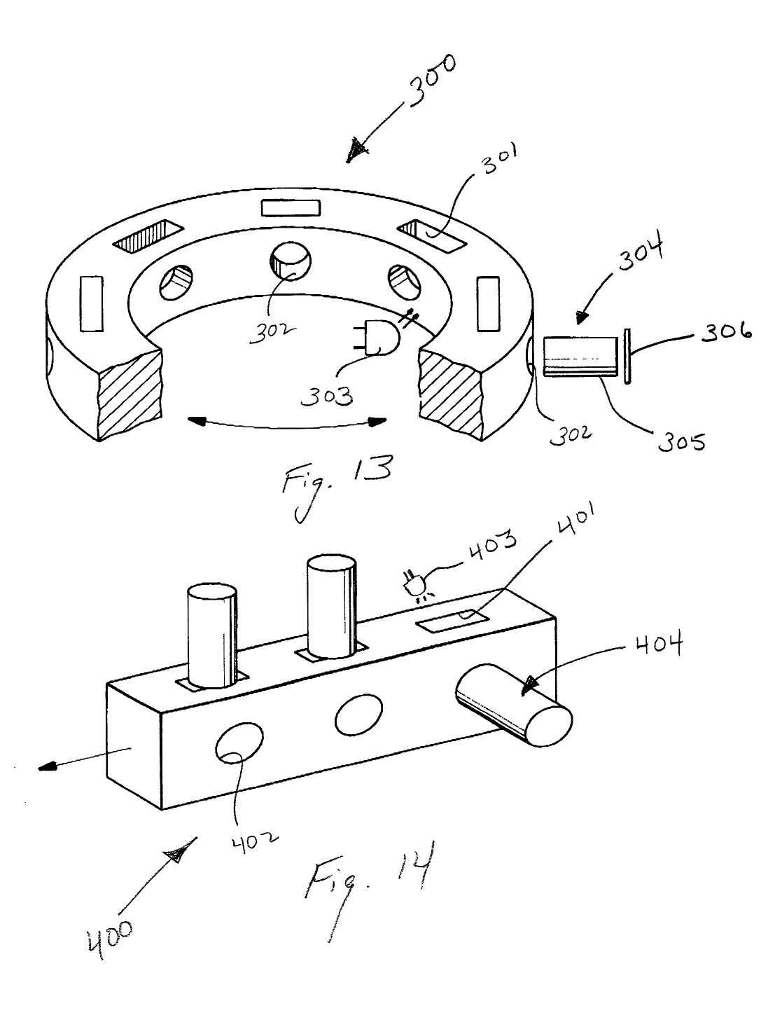

As described in U.S. patent application Ser. No. 09/450,963 (filed Nov. 30, 1999, and incorporated herein by way of reference) a variety of control systems and schemes may be used for regulating one or more conditions within well 214 housing an ovum or embryo. As shown in the schematic illustration of FIG. 11, embryo support assembly 212 may include a control system for regulating the environment in which the embryos are grown. Unlike prior art embryo growth methods wherein the embryo is transferred from one petri dish of fluid media to another, the embryo support assembly of the present invention allows the embryo to remain undisturbed within well 214 throughout the period of in vitro growth. As mentioned previously, the control system may comprise one or more heaters 254 configured for maintaining the temperature within well 214. As shown in FIG. 2, for example, heater 254 may be located in order to not only maintain the temperature of well 214, but also to maintain the temperature of fluid media within reservoir 224. In this manner, fluid media entering well 214 from reservoir 224 may be of approximately the same temperature as the fluid media already present within well 214, thus preventing thermal shock.

In order to more preccisely control the output of heater 254 and hence the temperature within well 214, a processor or other control device (or controller) may be provided for controlling the output of heater 254 and/or for regulating and/or monitoring other conditions within well 214. By way of example, this processor can comprise a CPU 296, however any number and variety of processors may be employed. CPU 296 may be configured for monitoring, analyzing, and/or controlling embryo growth conditions, and may operate in accordance with instructions stored, for example, in memory 297 (which may include both RAM and ROM). In one embodiment, CPU 296 is in electrical communication with heater 254, and may therefore be used to regulate the heat output therefrom (e.g., by regulating the amount of current delivered to heater 254 according to instructions stored in memory 297).

One or more sensors may also be included in embryo support assembly 212 in order to provide data indicative of conditions within well 214, or even within one or more fluid reservoirs. This data may be provided to CPU 296, and, in response to such data, CPU 296 may control the operation of the various electrical or electromechanical components of assembly 212 (e.g., fluid media pumps and valves, heaters, alarms, etc.). Furthermore, a visualization device such as CCD 278 may also provide data to CPU 296 for processing. Not only may the data from the various sensors and visualization device be used to regulate conditions within well 214, this data may be further processed and stored in, for example, memory 297. In this manner, an historical record of conditions within well 214, as well data indicative of the development of an embryo, may be later retrieved and analyzed.

Embryo support assembly 212 may also include a display device, such as a display 280 (e.g., a liquid crystal display screen). Display 280 may be configured for displaying any of a variety of information, such as the current temperature within well 214, various other well conditions, a clock indicating the amount of time the embryo has been developing in assembly 212, or even an image of the embryo itself. Thus, as shown in FIG. 11, display 280 may be in electrical communication with CPU 296 such that display data is output to display 280 by CPU 296.

As mentioned prevously, a visualization devise such as a CMOS imager or CCD 278 may be provided on the embryo support assembly in order to acquire one or more images of an embryo or ovum located within well 214. The embryo support assembly may be configured such that these images are acquired continuously or periodically, as desired. In addition, one embodiment of an embryo support assembly according to one embodiment of the present invention includes image manipulation and/or image recognition capabilities. While these features may be accomplished by CPU 296, it is also contemplated that a separate image processor may be provided (such as in the form of one or more additional CPU's or other processors or controllers of the type well-known to those skilled in the art). The embryo support assembly may be configured to process image date provided by the imaging device, such as altering the contrast, resolution, color, clarity, size or other aspects of each image, as desired, using techniques known to those skilled in the art.

The embryo support assembly may also incorporate image recognition capabilities, such as the ability to determine and/or analyze the symmetry, surface texture, wall thickness, edges or other imageable features of an embryo or ovum located in well 214. The imaging recognition capabilities may also include the ability to determine the presence or absence of various other particles which might be located within well 214, such as cellular debris, as well as the ability to recognize various imageable features of such particles. These imaging recognition capabilities may be provided by techniques well-known to those skilled in the art, such as, for example, the systems and methods described in U.S. Pat. No. 6,208,749 (which is incorporated herein by way of reference). It should also be pointed out that image data may be acquired using light of one or more different wavelengths. In particular, UV light may be used in acquiring images of an embryo or ovum located within well 214, as well as visible and/or IR light. These image processing and recognition capabilities may be encoded into memory 297, or even in a separate memory associated with an image processing system provided in the embryo support assembly according to one embodiment of the present invention.

Although the embryo support device of the present invention may be self-contained such that no external inputs are required for proper embryo development, interfaces 289 may be provided to allow for electrical communication between assembly 212 and an external device (such as an external computing device such as a PC, a computer network or server, or even a PDA). Any of a variety of well-known interfaces may be provided, such as simple electrical strip contacts, a network interface card, a USB port, or even a wireless interface for providing wireless communication between assembly 212 and an external device. In the embodiment shown in FIGS. 2 and 11, interface 289 comprises a plurality of conductive strips arranged for conductive engagement with a plurality of conductive members (e.g., male pins or strips) associated with an external device.

A variety of sensors may be provided in embryo support assembly 212, such as a temperature sensor 243 located within or adjacent to well 214. Additional temperature sensors may also be located at various other locations, such as within fluid media reservoir 224, with all of the temperature sensors in electrical communication with CPU 296. A suitable temperature sensor 243 may comprise, for example, a thermistor which is in electrical communication with CPU 296 (e.g., through an A/D convertor, not shown). In this manner, temperature sensor 243 will provide data to CPU 296 indicative of the temperature within well 214. A temperature set point may be stored in memory 297 and provided to CPU 296. Thus, for example, if the temperature within well 214 falls below the set point (as measured by temperature sensor 243), CPU 296 will send a signal activating or increasing the output of heater 254 in order to increase the temperature of well 214 according to, for example, control system instructions provided by memory 297.

It should also be pointed out that all of the sensors provided in the assembly may be formed directly on the substrate by well-known microfabrication techniques. For example, one or more of the sensors provided in assembly 212 may be fabricated using ion sensitive field effect transistor (“ISFET”) techniques well-known to those skilled in the art. One or more sensors may be integrally formed using such techniques, or individual sensors can be separately formed.

Various other types of sensors may also be provided, including one or more fluid level sensors within well 214 or reservoirs 224 or 254 in order to provide data indicative of the fluid level therein to CPU 296. If the fluid level falls below a predetermined set point, CPU 296 may activate an alarm in order to signal the user. Alternatively, if the fluid level within well 214 falls below a predetermined set point, CPU 296 may cause additional fluid media to be provided to well 214 from reservoir 224 by activating one or more valves and/or pumps configured for delivering fluid from reservoir 224 to well 214. In the exemplary embodiment of FIG. 11, a control valve 250 and a pump 252 are provided along channel 225 in order to allow for the controlled delivery of fluid media from reservoir 224 to well 214 (particularly under the control of CPU 296). Of course, the other fluid delivery techniques described previously may also be used.

Other types of sensors which may be provided, for example, within one or more of the fluid reservoirs, within one or more of the fluid channels, or within well 214, include: fluid flow meters (e.g., for indicating the amount of fluid urged into or out of well 214), pH sensors, oxygen sensors, carbon dioxide sensors, osmolarity sensors (for measuring the osmotic pressure of a fluid), pressure sensors, sensors for determining the concentration of one or more compounds or ions (such as calcium ions, sodium ions, nitrates or phosphates), or any other type of sensor which provides useful data concerning the fluid media and/or conditions within well 214 and/or the development of an embryo within well 214.

By way of further example, an oxygen sensor may be provided within well 214, with the sensor in electrical communication with CPU 296 in order to provide CPU 296 with an electrical signal indicative of the level of dissolved oxygen in the fluid media within well 214. Many fluid medias used for growing embryos are provided with a certain level of oxygen dissolved therein. Monitoring fluid oxygen levels can provide an indication of when a fluid media has aged or otherwise deteriorated (i.e., by a diminished level of dissolved oxygen). Thus, CPU 296 may cause fresh fluid media (or even oxygen) to be delivered to well 214 when a sensor detects a predetermined level of dissolved oxygen.

Sensors for detecting levels of urea, C0₂, ammonia, N₂, or other embryo waste products or materials which may otherwise be harmful to the embryo may be particularly useful in providing an indication that the fluid within well 214 needs to be changed. For example, CPU 296 may determine that, on the basis of signals from one or more such sensors and comparing those signals to one or more predetermined set points, elevated waste levels are present in well 214. When this occurs, CPU 296 may activate the fluid supply system in order to deliver (e.g., pump) new fluid media into well 214 and urge the old, waste-contaminated fluid media out of well 214 into reservoir 262. In this manner, the assembly of the present invention may ensure that the embryos do not remain in a potentially toxic environment for an extended period of time. Of course it is also contemplated that new fluid media may be urged into well 214 according to a predetermined schedule, and/or in response to embryo growth or other conditions within well 214, thereby also helping to remove potentially toxic waste away from each embryo.

As mentioned previously, one or more pH sensors may also be provided, for example, in well 214 and/or fluid media reservoir 224. Such fluid sensors will provide a signal to CPU 296 (or other type of processor or controller) which indicates the pH at the particular sensor's location. CPU 296 may, for example, compare such signals to one or more predetermined set points in order to determine whether or not the pH is appropriate. Using, for example, a feedback or feedforward control system, CPU 296 may adjust the pH within well 214 or reservoir 224 (or at whatever location the pH sensor is located) in order to return the pH to the appropriate value. The pH may be adjusted, for example, by delivering an acid, base or buffer solution to well 214 or reservoir 224 from, for example, an additional fluid reservoir not depicted in FIG. 11. Alternatively, embryo support assembly 212 may include the ability to generate ions appropriate for pH adjustment using electrolysis (such as one or more electrodes positioned within, or in fluid communication with, well 214). One or more membranes which only allow such ions to pass therethrough may also be provided, such that the generated ions may be selectively urged into well 214 or reservoir 224, as needed. Of course, other well-known techniques for adjusting pH may also be employed. Furthermore, the control system of the embryo support assembly may also be configured to control various other conditions within well 214, reservoir 224, or other portion of the support assembly using, for example, feedback or feedforward control schemes.

The embryo support assembly of the present invention may be provided to the end user in a sterile condition, with a suitable fluid media already in well 214, and additional fluid media (either the same or a different media than that in well 214) may be present in reservoir 224 (as well as in any additional fluid media reservoirs provided in the embryo support assembly). Prior to insertion of an embryo into well 214, it may be desirable to first ensure that the fluid within well 214 is at the proper temperature, particularly since embryo support assembly 212 may be provided to a user in an unpowered condition (e.g., the assembly is turned off to preserve power). Prior to embryo insertion, the user may activate assembly 212 (e.g., turn on) by means of a switch or other mechanism configured for user manipulation (e.g., an on/off button provided externally on apparatus 212). A switch 292 (see FIG. 11), for example, may be provided between power supply 290 and CPU 297.

A power switch may even be incorporated into valve 234. For example, one pole 241 of a switch may be provided on valve 234, as best seen in FIGS. 6 and 7, with another pole 242 provided in a region adjacent valve 234. Poles 241 and 242 may be configured such that when valve 240 is rotated (such as by means of valve handle 240 in FIG. 2), poles 241 and 242 will contact one another, thereby closing the switch and providing power to CPU 297 and/or other components of assembly 212.

By way of example, in FIGS. 2 and 6, valve 234 is positioned such that fluid communication is provided between the interior of well 214 and the ambient (through port 235), and poles 241 and 242 of the power switch are not in electrical communication with one another. In this position, access to the interior of well 214 may be obtained through port 235 and passageway 236 (e.g., for embryo insertion or removal), however the assembly is in an unpowered state. Before embryo insertion, however, valve handle 240 may be moved counterclockwise to the position of FIG. 7, thereby placing poles 241 and 242 in electrical communication with one another and providing electrical power to CPU 297 and other components. Assembly 212 may be preprogrammed (e.g., by means of instructions stored in memory 297) such that this initial “powering-up” will activate heater 254 in order to warm the fluid within well 214 (and reservoir 224) to a predetermined temperature suitable for embryo growth. Once the well temperature reaches the predetermined level, assembly 212 may notify the user. Such notification may be provided, for example, by a visual and/or audible alarm, a message on display 280, and/or even by simply displaying the temperature to the user on display 280. In fact, it is contemplated that display 280 may be used to display a series of messages to the user designed to provide step-by-step instructions for use.

After the well temperature has reached the predetermined level, the user rotates valve 234 back to the position of FIGS. 2 and 6, and may then insert one or more embryos into well 214 (e.g., by means of a catheter) through port 235. Although this may result in a temporary loss of power to CPU 296, assembly 212 may be programmed to “remember” that it has gone through the initial warming step prior to embryo insertion. Of course a variety of other configurations may be used in order to provide power to CPU 296 after initial warm-up. For example, instead of incorporating a power switch into valve 234, a separate power switch may be provided on assembly 212. In any event, after embryo insertion, valve 234 may be closed by rotating handle 240 clockwise so that valve 234 is in the position shown in FIG. 7. In this position, valve 234 provides fluid communication between reservoir 224 and well 214, thereby allowing fluid media to be urged from reservoir 224 into well 214.

Once the embryo has been inserted into well 214 and the system activated (e.g., by rotation of valve 234 and the resulting closure of the power switch), the control system of apparatus 212 may regulate and monitor the conditions within well 214. For example, CPU 296 may maintain the well temperature and pH at the desired level, and may even cause new fluid media to be urged from reservoir 224 into well 214. The conditions within well 214 as well as the delivery of new fluid media thereto may be controlled according to a predetermined schedule and/or in response to sensed conditions within assembly 212 and/or even the growth of the embryo itself (as determined, for example, by the image recognitioin system described previously). In this manner, embryo support assembly 212 provides an automated, self-contained environmental control system which may be configured for providing the optimal conditions for embryo growth and development.

Embryo support assembly 212 may not only provide the optimal growth and development conditions with minimal user intervention, these conditions also may be tailored to the specific needs of each individual embryo. By way of example, a series of images of the embryo may be acquired over a period of time (e.g., by using CCD 278), and these images may even be used to provide a time-lapse video of embryo growth. Assembly 212 may even be programmed so as to provide image recognition whereby, for example, cell counts of an embryo may be performed over time, thus providing a way of determining the progress of the development of the embryo. Alternatively or in addition, optical density measurements may be employed for monitoring the nuclear mass of the embryo. The development progress of the embryo (e.g., the speed of cell division) may in turn be used to adjust the conditions within well 214 accordingly, or even to select one or more embryos which have grown at a predetermined optimal rate. The images of the embryo may also be compared to a predetermined set of images which represent optimal morphology, thus allowing the conditions within well 214 to be adjusted accordingly and/or allowing the selection of one or more embryos which most closely match the optimal morphology. As further described herein, data concerning the development progress of the embryo (such as the rate of cell division) may even be used, in conjunction with data concerning the growth conditions within well 214 to determine which conditions achieve the optimal results. In this manner, the growth of future embryos may be further optimized based upon previously-acquired data.

Embryo support assembly 212 may even be shipped during the embryo growth process, since, in one embodiment, no external power sources or other inputs are needed in order to maintain embryo viability. This can be particularly advantageous in that, for example, fertilization of an ovum may be performed at one location, the fertilized ovum inserted into well 214, and assembly 212 thereafter shipped (e.g., by conventional shipping means such as an air, sea or land carrier) to an end user for further embryo growth and/or implantation.

Although the embryo support assembly according to some embodiments of the present invention is completely self-contained and requires no external connections to maintain embryo viability, the present invention also includes a cartridge configured for receiving one or more embryo support assemblies. The cartridge may be provided in a variety of configurations, with the cartridge having one or more receiving locations (such as a chamber) for receiving an embryo support assembly. These receiving locations may also have a variety of configurations, and may merely comprise a surface or other feature configured to receive, and preferably engage, an embryo support assembly.

In the exemplary embodiment of FIG. 12, cartridge 300 comprises a ring-shaped member having one or more compartments 301 which are sized and configured to receive an embryo support assembly 212 therein, as shown. Any number of compartments 301 may be provided, and each may be sized and configured such that an embryo support assembly 212 placed therein will be located entirely (or at least partially) within a compartment 301. In addition, each compartment 301 and/or each embryo support assembly 212 may be configured such that the embryo support assembly may be inserted into compartment 301 in a single, predetermined orientation.

Cartridge 300 also includes apertures 302 which may extend through the entire thickness of cartridge 300, with each aperture 302 intersecting one of the compartments 301. As more fully described below, apertures 302 allow for the visualization of an embryo housed within an embryo support assembly 212 which has been inserted into a cartridge 300. It is also contemplated that one or more lens elements may be positioned within one or more of the apertures 302 in order to provide for improved or even magnified visualization of the embryos. Alternatively, apertures 302 may comprise transparent regions in cartridge 300 through which the embryos may be visualized.

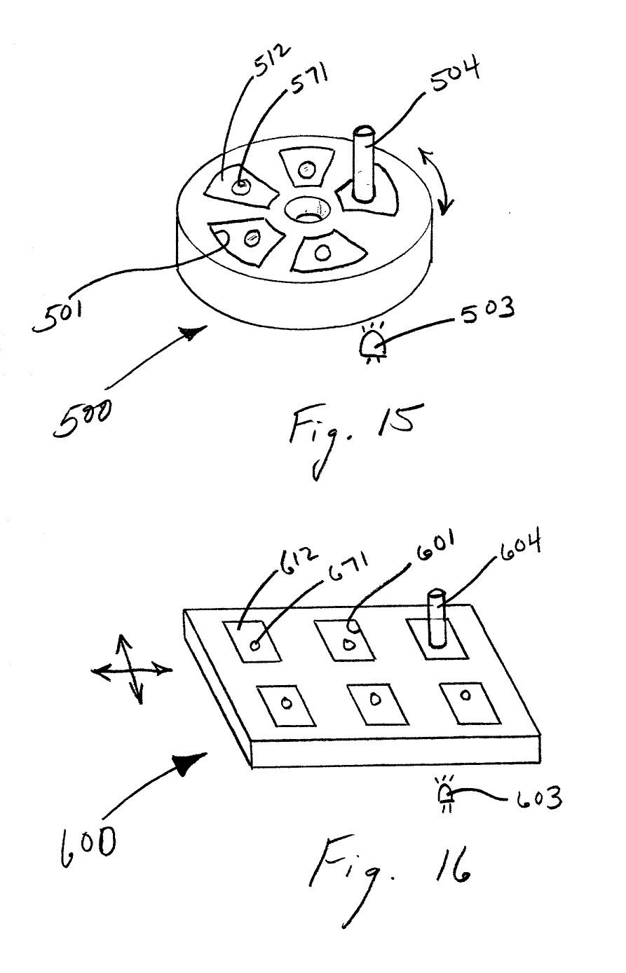

FIG. 13 is a perspective, cut-away view of cartridge 300. While each embryo support assembly 212 may include its own light source, and optionally its own imaging device, in other embodiments of the present invention embryo support assembly 212 does not include such features (or may include only a light source with no imaging device). Therefore, in order to visualize an embryo located within the well of an embryo support assembly 212, an external imaging device is located such that it may acquire an image of the embryo through lens or window 271 of the embryo support assembly. In many instances (particularly if there is no light source in the embryo support assembly), it may be also necessary to illuminate the embryo for proper visualization. The embryo may be front lit or back lit, as desired. In the case of back lighting, the region immediately behind embryo well 214 may be transparent or translucent so that light may be directed from the backside of the embryo support assembly into embryo well 214. Of course, front lighting may be provided simply by directing light through lens or window 271.

In the case of back lighting, cartridge 300 provides a convenient arrangement for providing such illumination. As seen in FIG. 13, a light source 303 may be positioned within the interior of ring-shaped cartridge 300 such that light emitted therefrom is directed through an aperture 302 into an embryo support assembly 212 located within the compartment 301 through which the aperture extends. In this manner, if embryo support assembly 212 is positioned within cartridge 300 such that lens or window 271 faces outwardly, and if embryo support assembly 212 is transparent or translucent in the region immediately behind embryo well 214, light emitted from light source 303 may be used to illuminate (back light) an embryo located within embryo well 214.

As also seen in FIG. 13, an imaging device 304 for acquiring an image may be positioned adjacent the exterior of cartridge 300 adjacent an aperture 302. Imaging device 304 may comprise any of a variety of well-known devices (such as a CCD or CMOS imager). For example, in the embodiment shown in FIG. 13, imaging device 304 comprises a lens system 305 (which includes one or more lens elements) and at least one light responsive sensor (such as a CCD 306). If light source 303 and imaging device 304 are located adjacent the same aperture 302, imaging device 304 can be used to visualize an embryo located within an embryo support assembly 212 positioned within the compartment 301 intersected by the aperture 302. In order to visualize an embryo located within a different embryo support assembly 212 (and hence in a different compartment 301), light source 303 and imaging device 304 are merely repositioned adjacent the aperture 302 intersecting the appropriate compartment 301. In this manner, for example, light source 303 and imaging device 304 may be simply rotated about the inner and outer circumference, respectively, of ring-shaped cartridge 300 in order to provide for the visualization of the embryo housed within each compartment 301.

It is also contemplated that, instead of advancing light source 303 and imaging device 304 about the inner and outer circumference of cartridge 300, the cartridge itself may rotate. Thus, light source 303 and imaging device 304 may remain stationary while cartridge 300 is rotatingly advanced so that light source 303 and imaging device 304 will be positioned adjacent the desired aperture 302. It should also be pointed out that light source 303 may remain stationary while imaging device 304 is advanced around the circumference of cartridge 300. Light source 303 may thus be configured to simultaneously illuminate the embryo well of each of the embryo support assemblies located within the compartments of cartridge 300.

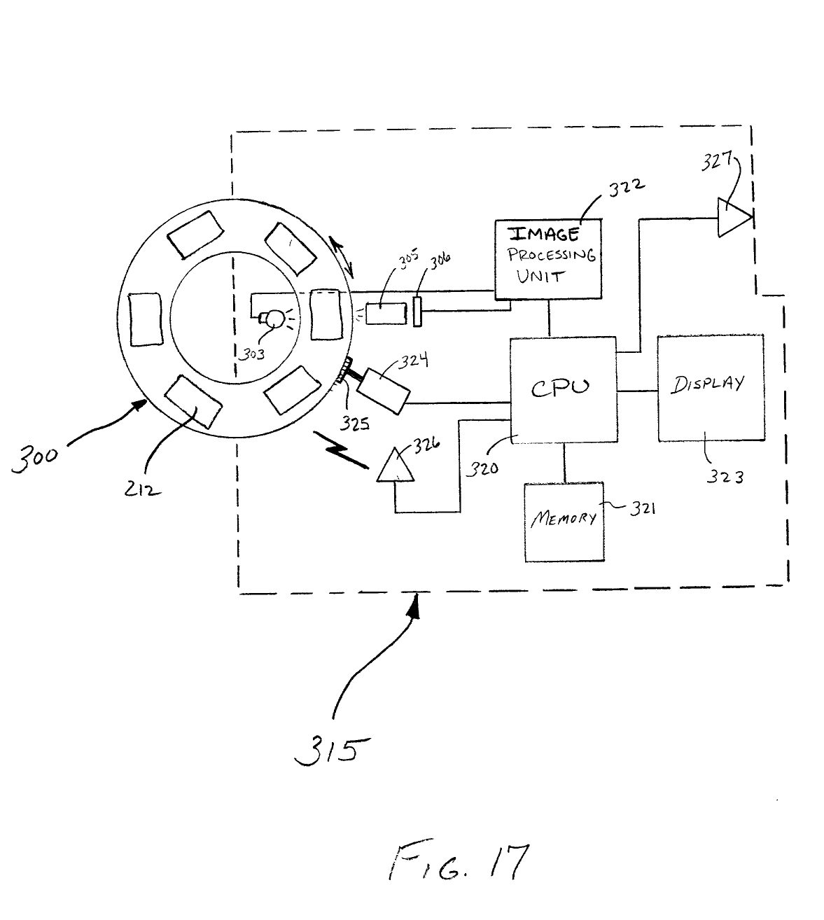

As mentioned previously, the cartridge of the present invention may be provided in a variety of configurations. Thus, as seen in FIG. 14, another embodiment of the present invention provides a cartridge 400 which has an elongated configuration with one or more compartments 401 for receiving embryo support assemblies arranged in a generally linear configuration. Once again cartridge 400 may also include apertures 402 which extend width-wise across cartridge 400 and intersect a compartment 401. A light source 403 may be located on one side of cartridge 400 adjacent an aperture 402, and an imaging device 404 located on the opposite side of cartridge 400 adjacent the same aperture. In this manner, an embryo housed with an embryo support assembly located within compartment 401 may be visualized (e.g., using imaging device 404). In order to visualize other embryo support assemblies within cartridge 400, light source 403 and imaging device 404 may be advanced along the length of cartridge 400. Alternatively, imaging device 404 and light source 403 may be remain stationary, while cartridge 400 is advanced.

FIG. 15 depicts yet another alternative embodiment for a cartridge according to the present invention, wherein cartridge 500 has a substantially disk-shaped configuration. Once again cartridge 500 includes one or more compartments 501 which are sized and configured to receive an embryo support assembly. The embodiment of FIG. 15 is particularly suited for use with embryo support assemblies having a horizontally-oriented embryo well. As seen in FIG. 15, embryo support assembly 512 has a lens or window 571 located on its upper surface, with lens or window 571 oriented to allow for visualization therethrough of an embryo housed within the well of embryo support assembly 512. Once again the region behind the embryo well of embryo support assembly 512 may be transparent or translucent in order to allow for back lighting of an embryo located within the well.

The lower surface of cartridge 500 may also include one or more apertures (not shown) located to allow light to be directed therethrough into the well of an embryo support assembly 512. Thus, as shown in FIG. 15, a light source 503 may be located adjacent an aperture in the bottom surface of cartridge 500 in order to direct light into a selected embryo well of an embryo support assembly 512. An imaging assembly 504 (e.g., a lens system and a CCD or CMOS imager) may similarly be located adjacent the lens or window 571 of the same embryo support assembly 512 in order to allow for imaging of an embryo housed therein (with back lighting provided by light source 503).

In order to visualize an embryo housed within another embryo support assembly 512, imaging assembly 504 and light source 503 may be simply advanced to the next location. Alternatively, as shown in FIG. 15, cartridge 500 may be rotated in order to align the desired embryo support assembly 512 with imaging device 504 and light source 503.

Yet another embodiment of a cartridge according to the present invention is shown in FIG. 16, wherein cartridge 600 has a generally planar configuration. Cartridge 600, like cartridge 500, may particularly be employed with an embryo support assembly 612 having a horizontally-oriented embryo well. A plurality of chambers 601 are provided for multiple embryo support assemblies, and chambers 501 may be arranged in a rectilinear array. Once again each embryo support assembly 612 may have a lens or window 671 through which an embryo housed within the well of the embryo support assembly 612 may be visualized. As seen in FIG. 16, an imaging device 604 may be positioned adjacent lens or window 671 of an embryo support assembly 612 for imaging purposes.

Like cartridge 500, the lower surface of cartridge 600 may include one or more apertures, each of which is located so as to be aligned with the embryo well of an embryo support assembly 612 positioned within a compartment 601 of cartridge 600. In this manner, a light source 603 may be used to back light the embryo for purposes of visualization using imaging device 604. As with the other embodiments of the cartridge of the present invention, imaging device 604 and light source 603 may be moved to the next embryo support assembly 612 in order to visualize another embryo. Alternatively, planar cartridge 600 may be moved in the manner shown by the arrows in FIG. 16 (i.e., along the x and y axis of the planar cartridge) in order to visualize an embryo within a selected embryo support assembly 612.

With respect to each of the embodiments for the cartridge described above, it will be understood that the individual embryo support assemblies may include an integral light source, as previously described. Therefore, a separate light source may not be necessary in order to visualize an embryo located within an embryo support assembly positioned within one of the cartridges described above.

As detailed above, an imaging device (and optionally a light source) may be employed with each of the various cartridge embodiments of the present invention. While a variety of apparatus may be employed for positioning the imaging device and optional light source with respect to the compartments of the various cartridge configurations described above, the present invention also provides a base assembly which is configured to accommodate one or more cartridges according to the present invention. The base unit may include, for example, various electronic componentry which not only allows for visualization (e.g., imaging) of embryos located within an embryo support assembly positioned within a cartridge, but also for receiving, storing and/or processing various data provided by each embryo support assembly. Thus, the base assembly of the present invention may be configured so as to be in electrical communication with each of the embryo support assemblies of a cartridge which is positioned within or otherwise attached to the base assembly.

FIG. 17 is a schematic illustration of one embodiment of a base assembly 315 according to the present invention. As noted in FIG. 17, a cartridge 300 may be positioned at least partially within base assembly 315, as shown. As more fully described herein, base assembly 315 may include one or more of the following: an imaging device (e.g., lens system 305 and CCD 306), a light source 303, an image processing unit 322, a CPU 320 (or other type of processor), memory 321 (RAM and/or ROM), one or more communication devices (e.g., 326 and 327), and a cartridge drive unit (e.g., a motor 324 and a driven gear 325).

When a cartridge 300 having one or more embryo support assemblies 312 positioned therein is inserted into base assembly 315, the imaging device of base assembly 315 may be employed to visualize the embryos housed within the embryo support assemblies 212. In the exemplary embodiment of FIG. 17, the imaging device may comprise a lens system 305 (comprising one or more lens elements) and at least one light responsive sensor such as CCD 306 (or other device suitable for acquiring an image such as a CMOS imager). If a light source is not provided within the individual embryo support assemblies 212, or if additional lighting is desired, base assembly 315 may also include a light source 303. Light source 303 may be configured and positioned such that when cartridge 300 is inserted into base assembly 315, light source 303 will be located within the interior of cartridge 300, as shown. In this manner, and as described previously, light source 303 may direct light into the embryo well of an embryo support assembly 212 for imaging purposes. An image of the embryo is acquired by CCD 306, and a signal representative of this image is transmitted to an image processing unit 322. The image processing unit will process the image signal received from CCD 306 and transmit a digital representation of an image of the embryo to CPU 320 for further processing, storage and/or display. Although image processing unit 322 is depicted as a separate unit within base assembly 315, image processing may also be performed within CPU 320 in accordance with instructions provided by memory 321.

After an embryo image has been acquired, it may be desirable to acquire an image of an embryo housed within another embryo support assembly 212 positioned within cartridge 300. As described previously, this may be accomplished merely by rotating cartridge 300 such that another embryo support assembly 212 is positioned adjacent the imaging device and light source of base assembly 315. A variety of mechanisms may be provided for advancement of cartridge 300. By way of example, a motor 324 (e.g., a stepper motor) may be employed to rotate cartridge 300. Rotational force provided by motor 324 may be transmitted to cartridge 300 in a variety of manners, such as by using a simple gear 325 which is driven by motor 324. The undersurface of cartridge 300 may also be provided with teeth which extend around the circumference of cartridge 300 such that these teeth may be engaged by gear 325. In this manner, rotation of gear 325 causes the indicated rotation of cartridge 300. Of course it will be understood that a variety of other mechanisms may be employed for causing the desired rotation of cartridge 300, such as alternative gearing arrangements, or even belt drive systems. It is also contemplated that cartridge 300 may be manually rotated, as desired, in order to allow for imaging of an embryo located within a selected embryo support assembly 212.

As mentioned previously, image processing unit 322 provides a digital representation of an image of an embryo to CPU 320. CPU 320 may then further process the image information, as desired, in accordance with instructions provided by, for example, memory 321. Base assembly 315 may also be configured to store the images in memory 321, as desired. Base assembly 315 may also include a display 323 (such as an LCD monitor or similar type of display unit). CPU 320 may therefore transmit each image to display 323 so that the image can be viewed on the display. Image processing unit 322 and/or CPU 320 may also perform the image processing and recognition functions described previously herein, as desired.

As mentioned previously, each embryo support assembly may include an interface 289 for providing communication between assembly 212 and another device (see FIG. 2). When a base assembly according to the present invention is employed, interface 289 may be configured to provide electrical communication between embryo support assembly 212 and base assembly 315. This may be accomplished by a variety of devices well-known to those skilled in the art. By way of example, cartridge 300 may be configured such that interface 289 of each embryo support assembly 212 positioned within cartridge 300 will be in electrical communication with cartridge 300 (e.g., by means of a plurality of conductive members such as male pins or strips which are in conductive engagement with interface 289 when the embryo support assembly is positioned within a compartment 301 of cartridge 300). Cartridge 300 may then be provided with its own interface which provides electrical communication between cartridge 300 and base assembly 315 when cartridge 300 is positioned within or otherwise attached to base assembly 315. In this manner, cartridge 300 may act as an intermediary between each embryo support assembly 212 and base assembly 315 for purposes of electrical communication.

Alternatively, and as depicted in FIG. 17, each embryo support assembly 212 may be configured for wireless communication with base assembly 315. Thus, a wireless interface 289 may be provided within each embryo support assembly 212. Base assembly 315 may also include a wireless interface 326 configured for electrical communication with wireless interface 289 of each embryo support assembly 212. Such wireless communication may be provided, for example, by means of RF or infrared signals.

Since each cartridge 300 may include multiple embryo support assemblies, base assembly 315 may be configured for either continuous or intermittent communication with each of the embryo support assemblies within cartridge 300. For example, it may be desirable to provide for communication between base assembly 315 and a single embryo support assembly 212 at any given time in order to reduce processing and memory requirements. Alternatively, base assembly 315 may be in continuous communication with each of the embryo support assemblies 212 of a cartridge located within base assembly 315. As yet another alternative, base assembly 315 may be configured to only periodically communicate with embryo support assemblies 212, or even only communicate with embryo support assemblies 212 upon the instruction of a user. For example, the user may instruct base assembly 315 (e.g., by using a suitable input device such as a keyboard, which is not shown) to communicate with one or more of the embryo support assemblies 212 within cartridge 300. It is also contemplated that base assembly 315 may be programmed (e.g., by means of software or other instructions stored in memory 321) to receive information from embryo support assemblies 212 according to a predetermined schedule. Base assembly 315, particularly CPU 320, may also be configured to perform some of the processing functions which were previously described as being performed by CPU 296 of the individual embryo support assemblies 212. Furthermore, CPU 320 is merely exemplary of one type of processor or controller which may be provided in base assembly 315, and multiple CPU's or other processors or controllers may similarly be provided therein.

As described previously, each embryo support assembly 212 may include a variety of sensors for acquiring and storing information concerning conditions within the well within which the embryo is located. This data can also be used to regulate the conditions within the well, such as by raising or lowering the well temperature and/or pH. Such acquired data, as well as any regulation of the conditions within the embryo well, may be stored within memory 297 of the embryo support assembly (see FIG. 11). It may also be desirable to transmit such information to base assembly 315 for further processing, storage and display. In this manner, base assembly 315 may even be used to acquire and store a history of various data concerning the conditions within the embryo well of each embryo support assembly, as well as a history of the growth and development of the individual embryos. Since base assembly 315 may acquire such data from multiple embryo support assemblies, a significantly greater compilation of embryo growth data can be obtained. Statistical analysis and the like can be performed on such data, so that, for example, the growth conditions for future embryos can be optimized by, for example, adjusting or modifying algorithms and the like included in the set(s) of instructions used to operate the control system of the embryo support assemblies.

Base assembly 315 can also be configured to transmit information to each embryo support assembly 212 in the same manner as which information is received. For example, base assembly 315 may transmit (i.e., download) new parameters, growth conditions, and even various programs for controlling the operation of each embryo support assembly 212. It will be apparent, therefore, that base assembly 315 can even be used to “reprogram” the computer control system contained within each embryo support assembly 212.

By way of example, data concerning the growth and development of each embryo (such as the rate of cell division, the generation of waste byproducts, etc.) may be acquired by base assembly 315, along with data concerning the conditions within each embryo well (such as temperature, pH, fluid media input to the well, etc.). Base assembly 315 may then compile and process such information in order to determine and evaluate the effect of various well conditions and other growth parameters on embryo development. This data may be analyzed by base assembly 315 in order to determine the optimal growth conditions and other parameters for embryo development and viability.

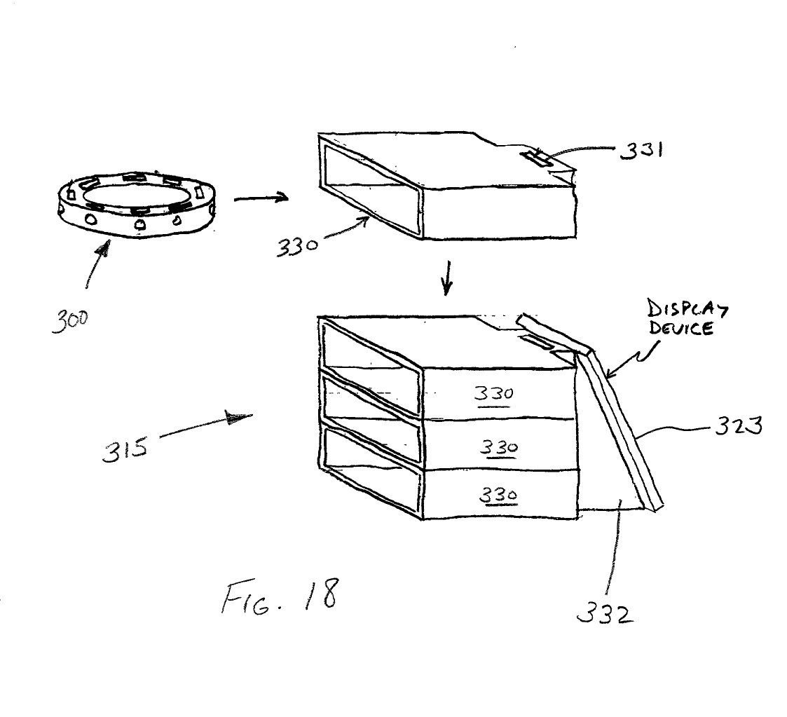

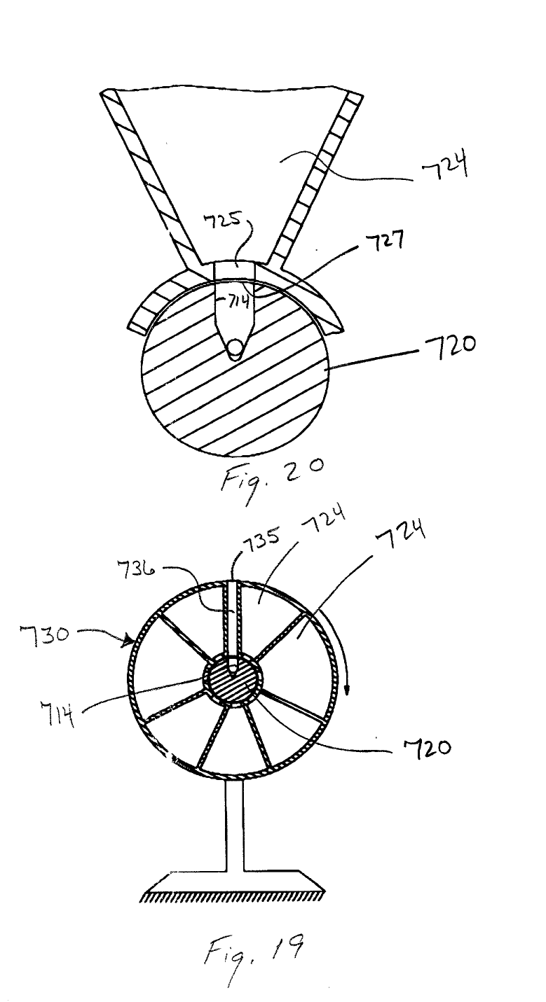

Although the individual embryo support assemblies will generally be preprogrammed to not only regulate conditions within the individual embryo wells, but also to, for example, schedule various changes in such conditions (such as the delivery of new fluid media to the embryo well), it may be desirable to modify such instructions in order to continually optimize embryo growth and viability. Therefore, base assembly 315 may be configured to process and analyze data acquired from embryo support assemblies and thereby refine the instruction set(s) used to regulate the conditions within each embryo well. This new instruction set may then be delivered to each embryo support assembly (e.g., through interface 289) such that the new instruction set will replace or modify the existing set of instructions stored within, for example, memory 297 of each embryo support assembly. In this manner, the set of instructions stored within each embryo support assembly may be continually or periodically replaced or revised in order to further optimize embryo growth and viability based upon data acquired from multiple embryo support assemblies. In addition, as further described herein, base assembly 315 can be configured to accommodate multiple cartridges 300, as shown in FIG. 18. In this manner, base assembly 315 may acquire data from multiple cartridges, each of which has multiple embryo support assemblies therein, thus allowing for even greater optimization of the instruction set stored within each embryo support assembly.

Even greater optimization may be achieved by configuring base assembly 315 to transmit data acquired from individual embryo support assemblies to an external, and even a remote, computing device, such as a centrally-located computing device (e.g., a server) configured to receive data from base assemblies located across the country or around the world. This centralized computing device may then analyze all of the acquired date in order to optimize the set of instructions used to operate the individual embryo support assemblies. The centralized computing device may then “download” new or revised instruction sets to one or more of the base assemblies (such as the base assemblies from which the original data was acquired). The base assemblies receiving such new or revised instruction sets may then transmit these instruction sets or revisions to the individual embryo support assemblies, as described above.

It is also contemplated that each base assembly 315, or one or more external computing devices receiving data from one or more base assemblies 315, may be programmed to perform further optimization procedures. For example, different instruction sets may be transmitted to each of the embryo support assemblies, such that each will regulate the conditions within the embryo well in a different manner. These instruction sets may be generated by the base assembly or the external computing device (such as a centralized computing device) in a random or partially-random manner in accordance with another set of programmed instructions. Such techniques will ensure that each embryo support assembly will operate in a slightly different manner so that the data acquired therefrom may be more effectively used for purposes of optimization.

For example, one instruction set delivered to an embryo support assembly may dictate that the embryo housed therein is grown at a temperature which is slightly different than the temperature dictated by the instruction set delivered to a second embryo support assembly. Data concerning the growth and viability of the embryo (such as the rate of cell division, or even characteristics determined by the image recognition capabilities described previously) may then be transmitted to the base assembly and/or the centralized computing device so that an analysis may be made as to the effect of the slight temperature difference on the growth and viability of an embryo. In this manner, the optimum temperature (or temperature profile over a period of embryo growth time) may be determined.

The base assembly and/or centralized computing device may even be configured such that subsequent data concerning the viability of the embryo (e.g., whether or not subsequent implantation of the embryo in the mother was successful) may be received. Such data may be received by base assembly 315 or a centralized computing device using, for example, an input device associated therewith, or even an input device configured to communicate with base assembly 315 or a centralized computing device (such as a PC which communicates with base assembly 315 or the centralized computing device over the Internet, or other means of communication).

As discussed previously, each embryo support assembly 212 may include its own power supply. However, when cartridge 300 is positioned within base assembly 315, base assembly 315 may be configured to provide or transmit the necessary power to each embryo support assembly 212, thereby preserving the individual power sources within each embryo support assembly 212. Thus, while each embryo support assembly 212 may be fully self-contained, base assembly 315 may provide further power and/or processing needs, as desired.

Base assembly 315 may further include a second interface 327 for providing electrical communication between base assembly 315 and an external device (such as another external computing device such as a PC or even a PDA). Once again, any of a variety of well-known electrical interfaces may be employed, such as those described previously. Interface 327 may even provide communication to an external computer network, such as the Internet, via a phone line, optical fiber (e.g., using a cable modem), or even a wireless connection. In fact, it is contemplated that base assembly 315 may be configured for communication over the Internet with a remote user and/or one or more remote, centralized computing devices. In this manner, a person located remotely from base assembly 315 may not only acquire data (including, for example, embryo images) from base assembly 315, but may also control the operation of base assembly 315 (e.g., instructing base assembly 315 to acquire an image of each embryo within cartridge 300). Thus, not only may a new instruction set be transmitted to the embryo support assemblies remotely by a computing device configured to optimize embryo growth conditions, instructions may be manually transmitted to the embryo support assemblies. Similarly, instructions may also be transmitted to each base assembly 315, based upon, for example, the analysis of acquired data or manual input from a user or operator.

It will be apparent that base assembly 315 may be configured such that an external user or operator may communicate with base assembly 315 through interface 327, thus allowing the remotely located user or operator to manipulate the operation of base assembly 315 (and even the individual embryo support assemblies 212). But, it may also be desirable to limit the ability of a remotely-located user to manipulate base assembly 315 and/or the individual embryo support assemblies, particularly if interface 327 provides for electrical communication across the Internet or other unsecure computer network. Thus, base assembly 315 may also be configured (i.e., programmed) to periodically or continuously transmit data (such as data received from the embryo support assemblies and embryo images) to an external server or other computing device. This external server or other computing device may be configured to be accessible through a network (such as the Internet), thus allowing remotely-located persons to acquire, view and even manipulate the acquired data (including images) transmitted from base assembly 315 without directly communicating with base assembly 315. Of course it is also contemplated that both configurations may be employed, whereby data is “uploaded” to a server or other computing device, while still providing the capabilty of direct access to base assembly 315 by an external user through interface 327. Passwords and various other types of security devices may be employed in order to control such direct access to base assembly 315 by a remotely-located user, while still allowing others to obtain or view the data uploaded by base assembly 315 (such as by persons interested in the condition or growth of a particular embryo).

By way of example, when the apparatus and systems of the present invention are used to grow human embryos, the parents of a growing embryo may wish to view an image of the embryo or other data concerning embryo growth and development. Thus, base assembly 315 may be configured to transmit such images and data not only to one or more centralized computing devices for analysis and optimization of the instruction set(s), but also to a second computing device (such as a server or other type of computer) which is configured to be accessible to the parents. In this manner, embryo images and perhaps other selected data, may be transmitted to a computing device which is accessible to the parents via, for example, the Internet or other computing network. Of course, this second computing device may still be configured to control access to such images and data using passwords and/or other types of security devices so that only the parents or other approved individuals may view the images and data, such an arrangement will still allow for the parents and other individuals to access the images and data without allowing them to manipulate, alter or otherwise affect base assembly 315, or the cartridges or embryo support assemblies located therein (other than, perhaps, instructing a base assembly or embryo support assembly to acquire an image of an embryo).

FIG. 18 is a perspective view of one particular embodiment of a base assembly 315. In the embodiment of FIG. 18, base assembly 315 comprises one or more docking stations 330, each having a suitable electrical connector such as a bus connector 331. The term “bus connector” simply refers to any type of electrical connector through which data or other signals may be transmitted from one device attached to the bus to another device which is also attached to the bus. Each docking station 330 is configured to receive an individual cartridge 300, as shown. Bus connector 331 is configured such that when one docking station is attached to another (such as by stacking one on top of another), the bus connectors of each docking station 330 will engage one another in order to provide electrical communication between the two docking stations. In this manner, multiple docking stations can be electrically connected to each other in order to provide a base assembly 315 which accommodates any number of cartridges 300.

Each docking station 330 may include all of the componentry identified in FIG. 17 for base assembly 315. Alternatively, some of the components of base assembly 315 may be shared by multiple docking stations, particularly since the bus connectors 331 provide electrical communication between adjacent docking stations. For example, each docking station may include an imaging device (e.g., lens system 305 and CCD 306), a light source 303, and a cartridge drive assembly (e.g., stepper motor 324). Base assembly 315 in FIG. 17 may also include an interface module 332, wherein interface module 332 includes CPU 320, memory 321, image processing unit 322, display device 323, and interfaces 326 and 327.

Interface module 332 may be configured such that one or more docking stations 330 may be attached thereto in electrical communication therewith. In this manner, for example, CPU 320 may be used to control the operation of multiple docking stations. In addition, as best seen in FIG. 18, it is also contemplated that not all of the docking stations need be in direct electrical communication with interface module 332. For example, interface module 332 may be configured such that it can be placed in direct electrical communication with a single docking station 330 (such as the lowermost docking station 330 depicted in FIG. 18). When additional docking stations 330 are stacked on top of each other, or otherwise attached in electrical communication to one another, bus connectors 331 of each docking station 330 will provide for indirect electrical communication between subsequent docking stations and interface module 332. In this manner, any number of docking stations may be attached to a single interface module, as desired.

It should also be pointed out that an input device may be provided on base assembly 315 (not shown). For example, a keyboard may be provided adjacent display device 323. Alternatively, display device 323 may even comprise a touch-sensitive display, thus integrating the input device and the display device as a single unit, as is well-known to those skilled in the art.

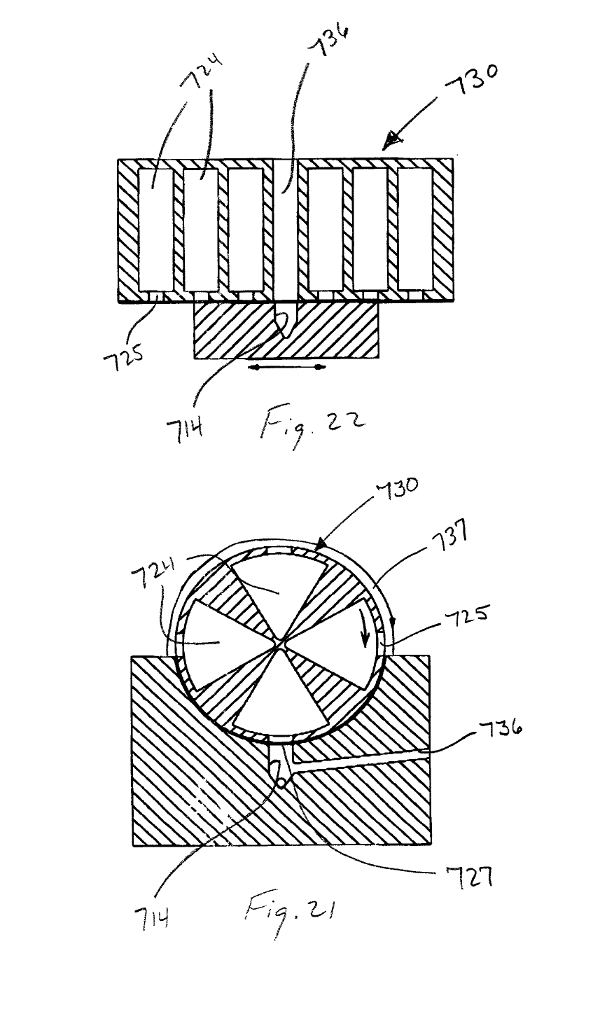

FIGS. 19-23 depict alternative embodiments for the embryo well and fluid reservoir components of an embryo support assembly according to the present invention. In particular, these arrangements may be used, for example, in place of the embry well and fluid reservoir arrangement of the embodiment shown in FIG. 2. For example, the arrangement shown in FIG. 19 can be used in place of embryo well 214, fluid media reservoir 224 and fluid waste reservoir 262 of embryo support assembly 212, as shown in FIG. 2. In general, each of the embodiments shown in FIGS. 19-23 include a plurality of “stations” which may be selectively brought into communication with the embryo well. Each station may comprise, for example, a fluid reservoir containing any of a variety of fluid media, or even a simple passageway through which an embryo may be inserted or removed from the embryo well. One of the stations may even comprise a passageway configured for fertilization of an ovum located within the embryo well (e.g., using an ICSI device).

In particular, one or more of the stations may comprise fluid media reservoirs. In the embodiments shown, the fluid volume of the embryo well is considerably smaller than the volume of the fluid media reservoirs. Therefore, when a particular media reservoir is brought into communication with the embryo well, the fluid media from the reservoir will diffuse throughout the embryo well in order to “bathe” the embryo with the new fluid media. Although a small portion of any previous fluid will generally remain in the embryo well, that fluid will be quickly dispersed throughout the entire volume of the media reservoir brought into communication with the embryo well, thereby minimizing any effects caused by this “old” fluid.

In the embodiment of FIG. 19, embryo well 714 may once again be provided in a tapered configuration, such that an embryo located therein will tend to be located near the base point of the well (particularly under the force of gravity when embryo well 714 is in the orientation shown in FIG. 19). Embryo well 714 is provided in a central hub 720. Hub 720 may be provided, for example, in a cylindrical configuration. A plurality of fluid media reservoirs 724 are positioned about the circumference of hub 720, as shown. Fluid media reservoirs 724 may be attached to, or even integrally formed with, one another, thus providing a cylindrical fluid supply assembly 730 having hub 720 at its center.

As seen in the enlarged cross-sectional view of FIG. 20, each fluid media reservoir 724 includes a fluid aperture 725 at its base. Embryo well 714 also includes a fluid aperture 727 at its upper end. Fluid aperture 725 on each fluid media reservoir 724 is configured such that when aperture 725 is located adjacent aperture 727 on embryo well 714, fluid communication is provided between fluid media reservoir 724 and embryo well 714. In this manner, fluid media within reservoir 724 will be provided to embryo well 714.

In addition to a plurality of fluid media reservoirs 724, fluid supply assembly 730 may also include one or more passageways 736 which, when aligned with embryo well 714, provide communication between the interior of well 214 and the ambient. In this manner, passageway 736 acts similarly to passageway 236 shown in FIG. 2. For example, passageway 736 may be used to insert an embryo into, or remove an embryo from, well 714 in the manner described previously. More than one passageway 736 may be provided. For example, separate passageways 736 may be provided for embryo insertion and removal.

After an embryo has been inserted into well 714 through port 735 on passageway 736, fluid supply assembly 730 may simply be rotated to bring one of the fluid media reservoirs 724 into fluid communication with embryo well 714 (as seen in FIG. 20). Once fluid communication is provided, the fluid media within reservoir 724 will enter well 714 (e.g., under the force of gravity), thus “bathing” the embryo in the fluid media. Thereafter, at a predetermined or selected time, fluid supply assembly 730 may be rotated further in order to bring a different fluid media reservoir 724 into fluid communication with embryo well 714. Advancement of fluid supply assembly 730 may be performed manually, or the embryo support assembly may be configured (e.g., programmed), to rotate fluid supply assembly 730 at a predetermined time (e.g., based upon a predetermined schedule and/or based upon the actual development of the embryo.) This process may be repeated for each of the fluid media reservoirs 724, as desired.

In the embodiment of FIG. 21, embryo well 714 is located adjacent to the exterior of fluid supply assembly 730. In this embodiment, each fluid media reservoir 724 has a fluid media aperture 725 located along the outer periphery of fluid supply assembly 730. In addition, rather than providing a passageway between the ambient and embryo well 714 through fluid supply assembly 730, passageway 736 is located externally from fluid supply assembly 730. Thus, the embryo may be inserted and removed from well 714 through passageway 736 in the manner described previously. Once the embryo is located within embryo well 714, fluid supply assembly 730 is simply rotated as shown in order to bring a fluid media reservoir 724 into fluid communication with embryo well 714 through fluid aperture 725. Once fluid communication is established, the fluid media will pass from the fluid media reservoir 724 into embryo well 714. At a predetermined or selected time thereafter, fluid supply assembly 730 is once again rotated in order to bring a new fluid reservoir 724 into communication with embryo well 714, thereby exposing the embryo to a different fluid media. A seal 737 (see FIG. 21) may be provided about fluid supply assembly 730, as shown, in order to prevent the escape of fluid. Similarly, a seal or other closure device may be provided at the external end of passageway 736 to further prevent fluid escape.

FIG. 22 depicts yet another alternative embodiment, wherein the fluid media reservoirs are arranged in a linear fashion along the length of a fluid supply assembly 730. In this embodiment, a passageway 736 is provided in fluid supply assembly 730 in order to provide communication between embryo well 714 and the ambient. However, in order to bring a new fluid media reservoir into communication with embryo well 714, fluid supply assembly 730 and/or embryo well 714 are moved in the direction shown (i.e., fluid supply assembly 730 and/or embryo well 714 are moved linearly with respect to one another.)

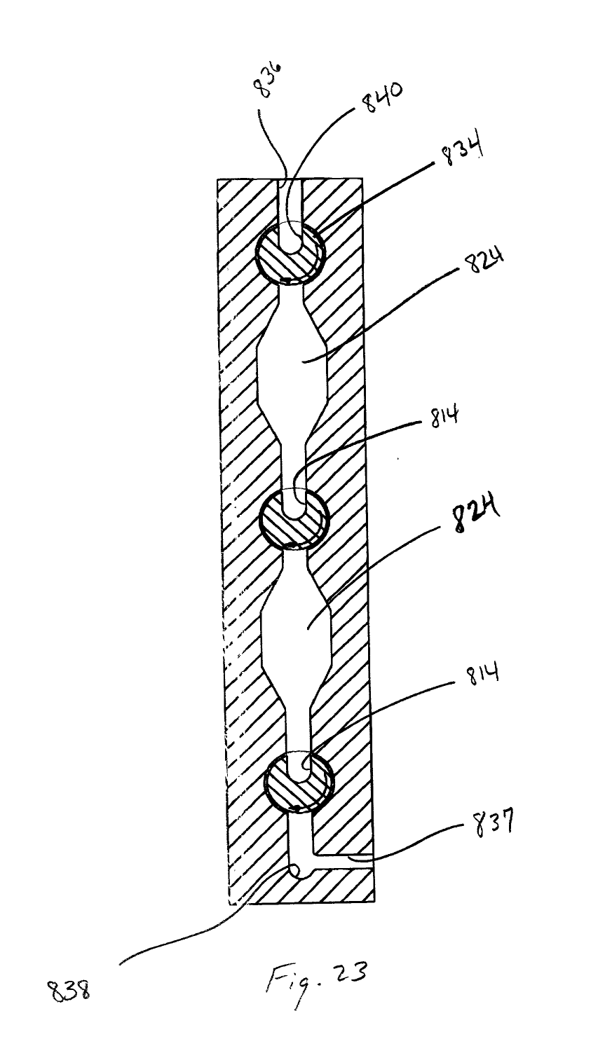

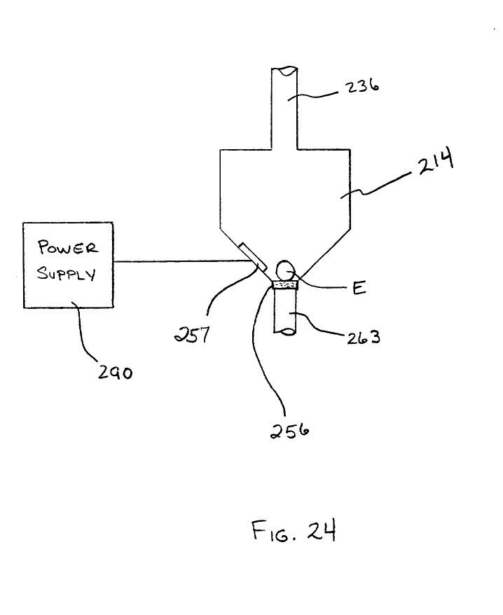

The embodiment shown in FIG. 23 is somewhat different than that shown in FIGS. 19-22, particularly in that multiple embryo wells are provided for a single embryo. In particular, a plurality of embryo wells 814 are provided. Each embryo well 814 may be shaped and configured as previously described so that the embryo will tend to remain at the center base portion of embryo well 814. In the embodiment of FIG. 23, however, each embryo well is rotatable in order to transfer an embryo from one well to another. In addition, each embryo well is in fluid communication with a different fluid media reservoir, thereby providing a system by which an embryo may be exposed to different fluid medias.

As shown in FIG. 23, each embryo well 814 is not only located at the base of a fluid media reservoir 824, but is also located above a second fluid media reservoir 824. Each embryo well 814 is movable between an embryo holding position wherein the upper open end of well 814 faces upwardly, and an embryo releasing position wherein the open end of well 814 faces downwardly. In the embodiment shown in FIG. 23, each embryo well 814 may be advanced from its holding position to its embryo releasing position simply by rotating the embryo well in the direction shown. When an embryo is located within an embryo well 814 that is positioned at its embryo holding position (FIG. 23), the embryo will be exposed to the fluid media contained within fluid media reservoir 824 located immediately above the embryo well. At a predetermined or selected time, the embryo well may be rotated as shown, thereby moving well 814 to its embryo releasing position. The embryo well will then be in fluid communication with the fluid media reservoir 824 located directly beneath well 814, and the embryo will fall downwardly (under the force of gravity) through the lower fluid media reservoir. In this manner, the lower fluid media reservoir acts as a passageway through which the embryo travels to the next well. At the base of the second fluid media reservoir, the next embryo well 814 is positioned in its embryo holding position, such that as the falling embryo reaches the base of the fluid media reservoir, it will simply fall into the next embryo well 814. In this manner, the embryo will now be exposed to the fluid media in the lower fluid media reservoir 824.

In order to allow for insertion of an embryo into the system of FIG. 23, an upper passageway 836 is provided, and is in communication with embryo receiving well 840. Embryo receiving well 840 may be similar to embryo wells 814, and thus may be provided on a rotatable body 834. In this manner, after an embryo has been inserted into embryo receiving well 840, body 834 is simply rotated as shown in order to rotate embryo receiving well 840 downwardly. Once embryo receiving well 840 is in communication with the upper fluid media reservoir 824, the embryo will simply fall downwardly through the fluid media until it reaches the first embryo well 814.

As for removal of the embryo from the system of FIG. 23, the lowermost embryo well may simply be rotated in the direction shown such that the embryo will be released downwardly into a lower catch basin 838. At this point, the embryo can then be removed through passageway 837.

Although the embodiments shown in FIGS. 19-23 may be employed with the embryonic support assembly described previously herein (particularly that shown in FIG. 2), the embryo well and fluid media reservoir configurations shown in FIGS. 19-23 may also be employed in other embryo support systems (e.g., embryo support systems which are not self-contained).

FIG. 24 is a schematic illustration of an alternative arrangement for embryo well 214. The arrangement of FIG. 24 may be used, for example, in the embryo support assembly shown in FIG. 2. In the embodiment of FIG. 24, embryo well 214 once again includes tapered side walls which help to ensure that an embryo E will tend to be located at the lower-most central region of embryo well 214, as shown. The embodiment of FIG. 24 differs from that shown in FIG. 2, however, in that waste fluid channel 263 enters well 214 adjacent the base thereof. In this manner, when new fluid media is urged into well 214 through passageway 236, the fluid media present within well 214 will be urged out of the well through waste fluid channel 263. A porous wall such as a filter 256 is located at the base of embryo well 214 adjacent waste fluid channel 263, such that channel 263 is in fluid communication with well 214 through filter 256. Filter 256 is configured to allow the passage of fluid media therethrough, along with cellular debris (such as cumulus cells) and other waste particles present in embryo well 214, but prevent the passage of the embryo therethrough. Thus, as shown in FIG. 24, the embryo may essentially be positioned on top of filter 256. Thus, filter 256 provides embryo well 214 with a porous wall through which fluid media may pass. In addition, one or more valves may be provided on waste fluid channel 263 in order to control the flow of waste fluid therethrough.

Unfertilized eggs are typically surrounded by cumulus cells (a form of cellular debris) which may inhibit fertilization of the egg. Therefore, if fertilization is to be performed in the embryo support assembly, it may be desirable to remove all or a portion of the cumulus cells. In addition, cumulus cells may remain around the exterior surface of a fertilized egg (i.e., an embryo) and it may be desirable to remove these cumulus cells from the embryo. Other particles and cellular debris may also be present within well 214, and a means for removing such particles and debris may be desired. While pulsing fluid media through well 214 may facilitate removal of cumulus cells and other debris, one embodiment of the present invention includes a device configured for removing cumulus cells and other debris from the oocyte or embryo. While such a device may comprise a simple mechnical agitator configured to agitate the fluid within well 214 in order to dislodge the cumulus cells, an acoustic wave generating device configured for propagating a plurality of acoustic waves through the interior of well 214 may alternatively be employed. In the embodiment of FIG. 24, this acoustic wave generating device comprises a piezoelectric element 257 which may be located in or adjacent to embryo well 214 for generating acoustic waves therein. In the embodiment of FIG. 24, piezoelectric element 257 is secured to an interior wall of embryo well 214, such as a wall located adjacent to the embryo. Alternatively, the piezoelectric element may be located adjacent or affixed to the exterior surface of a wall of embryo well 214, or may even form all or a portion of one of the walls of embryo well 214.

When a voltage is applied to a piezoelectric element, the element will change dimensions. By applying the proper voltage to piezoelectric element 257, particularly by applying an AC voltage, the piezoelectric element may be made to vibrate. Such vibration may be employed to generate a plurality of acoustic waves through the fluid media within embryo well 214, particularly ultrasonic acoustic waves. The acoustic waves generated within well 214 will cause the cumulus cells to separate from each other and from the oocyte (egg) or embryo. At the same time the acoustic waves are generated within well 214, and/or thereafter, fluid media may be urged into well 214 in order to flush the cumulus and other debris separated from the oocyte or embryo into waste fluid channel 263. In this manner, the cumulus cells and other debris can be effectively removed from the oocyte or embryo. Of course a variety of other devices may also be used in place of piezoelectric element 257 in order to generate such acoustic waves, particularly ultrasonic acoustic waves.

FIGS. 25-30 depict yet another alternative embodiment for a self-contained embryo support assembly. In some respects, however, the device shown in FIGS. 25-30 combines certain features of the single-well embryo support assemblies described above and the various cartridge embodiments designed to accommodate such single-well embryo support assemblies therein. Although one skilled in the art, after reading the present application, would recognize that the single-well embryo support assemblies described previously may be modified to provide multiple embryo wells and/or multiple fluid media reservoirs (and such modifications are well within the scope of the present invention), the device of FIGS. 25-30 is an alternative configuration for such a device.

It should initially be noted that, for purposes of clarity, certain features of the embryo support assembly 900 are not shown in FIGS. 25-30. For example, the device shown in these figures may be self-contained in that it includes a power source which is not depicted. Similarly, the various electronic components such as one or more CPU's (or other type of processors or controllers), memory, heaters, sensors, display devices, switches (such as a switch for energizing the device) and electrical interfaces are not shown in FIGS. 25-30 for purposes of clarity. However, the present invention contemplates that one or more (or even all) of these features may be included in the device shown in FIGS. 25-30. In addition, the embryo support device of FIGS. 25-30 may be readily fabricated using the microfabrication techniques described previously herein. In fact, as further described below, the device shown, for example, in FIG. 25 may be readily fabricated from two or more substrates which are, for example, micromachined, microembossed or micromolded to form the various features of the device.

Embryo support device 900 shown in FIG. 25 may comprise a disposable lower layer 901, and a reusable upper layer 902. Of course it is also contemplated that both of these layers may be disposable or reusable, as desired. Furthermore, although disposable lower layer 901 is depicted as being formed from two substrate layers, it will be apparent to one skilled in the art that a single layer, or even more than two layers, may be employed. Embryo support device 900, as depicted in FIG. 25 is configured to accommodate up to six embryos therein, and therefore includes six individual embryo wells. As further described herein, the particular embodiment shown in FIGS. 25-30 for embryo support device 900 also includes three fluid media reservoirs for each individual embryo well, as well as a separate fluid waste reservoir, for each embryo well. Of course, it is also contemplated that one or more common fluid media reservoirs and one or more common waste fluid reservoirs may be provided in place of the individual reservoirs described further herein. In addition, the provision of six embryo wells is merely arbitrary, since any number of embryo wells and fluid reservoirs may be provided, as desired.

Embryo support device 900 is substantially disc-shaped, however any of a variety of other shapes and configurations may also be employed. Disc-shaped embryo support device 900 also includes a bore which extends through the center portion of both the upper and lower layers 902 and 901, respectively, and is configured to receive a latch member 903. Latch member 903 is configured to lockingly secure lower layer 901 to upper layer 902 during use, and may be manipulated in order to release lower layer 901 from upper layer 902 as needed. Latch member 903 may accomplish this locking feature in any of a variety of well-known ways, and this locking feature is not explicitly shown in the figures. By way of example, the shaft portion 904 of latch member 903 (see FIG. 29) may be threaded, with similar threads provided on the interior of central bore 905A which extends through lower layer 901. In this manner, latch member 903 may simply be rotated such that the threads on shaft 904 will engage the threads on the interior surface of bore 905A, thus locking lower layer 901 to upper layer 902. The two layers may be unlocked from one another merely by rotating latch member 903 in the opposite direction.

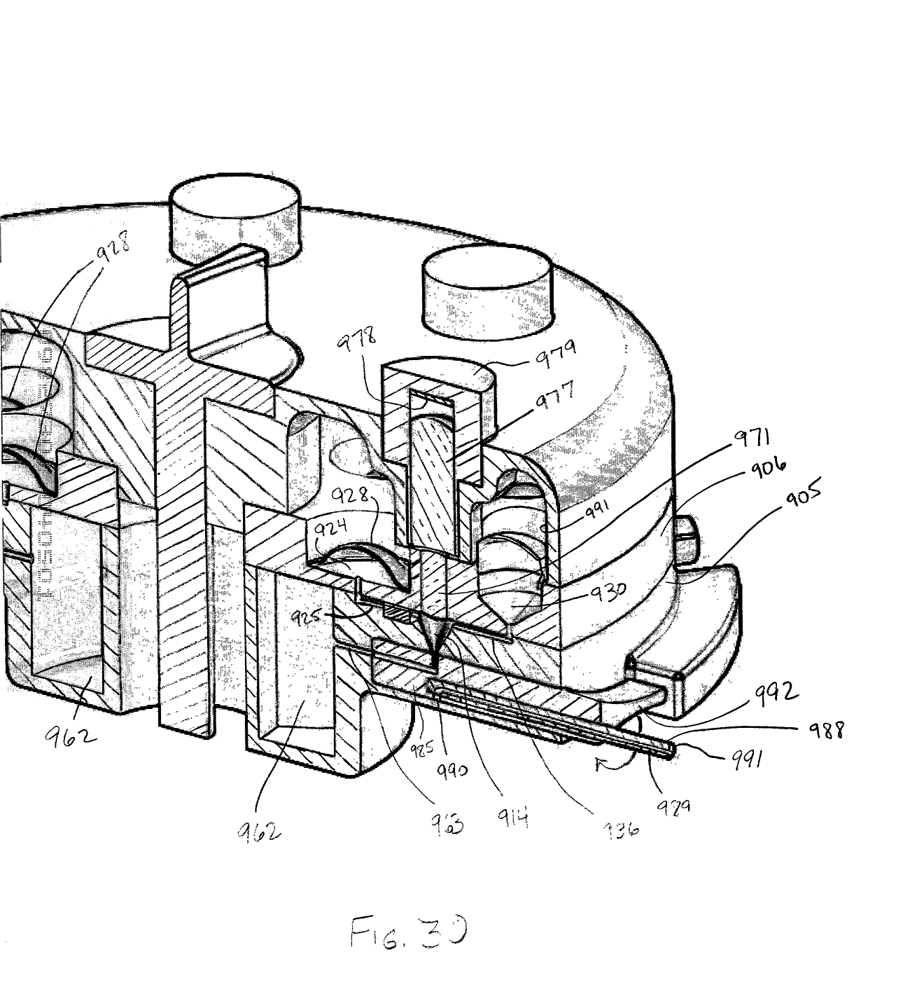

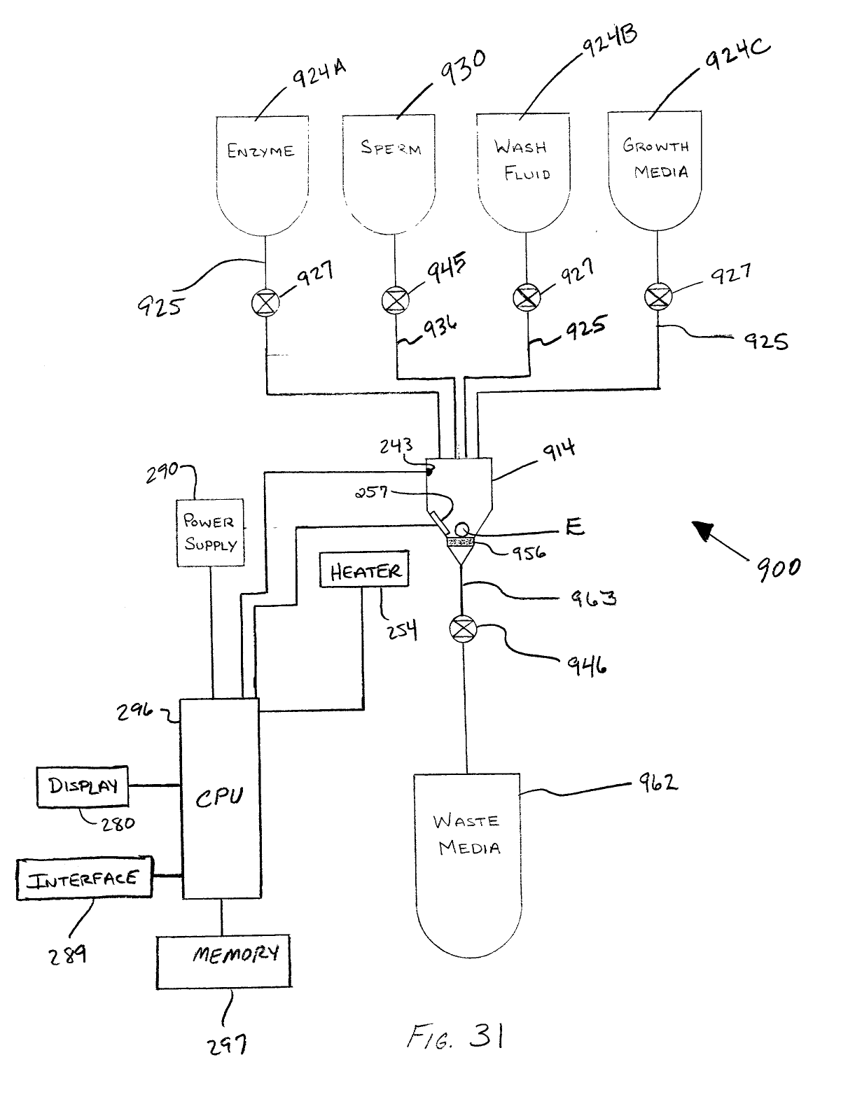

As best seen in the exploded view of FIG. 29, a plurality of embryo wells 914 are circumferentially located on first substrate 905 of lower layer 901. Each embryo well 914 may be formed, for example by micromachining, microembossing and/or micromolding first substrate 905. In addition, the number and arrangement of embryo wells 914 depicted in FIG. 9 is merely exemplary of one possible embodiment. As best seen in the cross-sectional views of FIGS. 28 and 30, each embryo well may be shaped similarly to that previously described. As shown, for example, embryo wells 914 may have tapered sidewalls such that an embryo located therein will tend to be located at the central base portion of embryo well 914 due to the force of gravity. A plurality of fluid media input channels 925 are also formed in first substrate 905, and are in communication with the interior of embryo well 914. Since the particular embodiment shown includes three fluid media reservoirs for each individual embryo well, three such fluid input channels 925 are provided for each embryo well 914. Although any of a variety of mechanisms and devices may be used to urge fluid media through channels 925 into embryo well 914, as shown in FIG. 28, a control valve 927 may be provided on each input channel 925. In this manner, valve 927 may be used to control the flow of fluid through each channel 925 into embryo well 914.

As best seen in FIGS. 28 and 30, an input channel 936 is also provided on first substrate 905 for each embryo well 914. As further described below, input channel 936 may be used to insert an oocyte, sperm or an embryo into embryo well 914. Therefore, input channel 936 is in fluid communication with the interior of embryo well 914, as shown. A waste fluid media reservoir 962 is also provided in first substrate 905 for each embryo well 914. In the particular embodiment shown, these waste fluid media reservoirs extend circumferentially around bore 905A and first substrate 905. A waste fluid media channel 963 is also provided, and may be configured to provide fluid communication between embryo well 914 and waste fluid media reservoir 962.

While each waste fluid media channel 963 may provide direct communication between well 914 and reservoir 962, the embodiment shown in FIGS. 28 and 30 further includes a transfer sleeve 985 through which fluid communication is provided. In particular, each transfer sleeve 985 includes a waste fluid media channel 986 which may be aligned so as to provide fluid communication between waste fluid media channel 963 and embryo well 914. In addition, a filter 956 or other porous material may be located between embryo well 914 and waste fluid media channel 986 of transfer sleeve 985, as best seen in FIGS. 28 and 30. Filter 956 will prevent the embryo from escaping well 914 into channel 986, but may be configured to allow the passage of not only fluid media, but also cumulus cells and other debris into channel 986. In fact, the central lowermost portion of each embryo well 914 may include an open aperture such that the embryo will generally be located immediately above filter 956 (and even supported thereon), with filter 956 providing a porous wall of well 914.

In the embodiment shown, transfer sleeve 985 is rotatably positioned within a bore extending through first substrate 905. Transfer sleeve 985 may further include a bore 987 configured to accommodate a transfer catheter 988 therein. Transfer catheter 988 includes a central passageway 989 through which an embryo may be extracted from an embryo well 914. When transfer catheter 988 is inserted into bore 987 of transfer sleeve 985, the proximal end 991 of transfer catheter 988 will provide fluid communication between the exterior environment and central passageway 989.

The distal end of transfer catheter 988 is configured such that when transfer catheter 988 is inserted into bore 987 of transfer sleeve 985 in the manner shown in FIGS. 28 and 30, the distal end of central passageway 989 will be aligned with a port 990 provided on transfer sleeve 985. Port 990 is configured to provide fluid communication between the exterior of transfer sleeve 985 and the interior of central passageway 989 on transfer catheter 988. In addition, port 990 is further configured such that when transfer sleeve 985 is rotated in the manner shown in FIG. 30, such as by using lever 992, port 990 will be brought into fluid communication with embryo well 914 at the base thereof. Thus, when transfer sleeve 985 is rotated in this manner, fluid communication will be provided between embryo well 914 and the external environment through central passageway 989 of transfer catheter 988. An embryo may thus be removed from embryo well 914 through central passageway 989.

Although an additional catheter or other device may be inserted into central passageway 989 for removal of an embryo from an embryo well 914, the device shown in FIGS. 28 and 30 is configured such that when transfer sleeve 985 is rotated in the manner described above, an embryo located within well 914 will fall, under the force of gravity, into central passageway 989 of transfer catheter 988 and generally remain therein. Transfer catheter 988 may then be removed from transfer sleeve 985, with the embryo remaining within central passageway 989 of the transfer catheter. This allows for easy removal of an embryo without requiring any physical manipulation of the embryo by the user.

Although first layer 901 of embryo support device 900 may comprise a single substrate layer, the embodiment shown in FIGS. 25-30 employs first and second substrates 905 and 906 for first layer 901. As best seen in FIG. 29, a plurality of fluid media reservoirs 924 are formed in second substrate 906. In the particular configuration shown, three fluid media reservoirs 924 are provided for each embryo well 914, and are configured such that fluids located therein may be provided to the embryo well with which the reservoirs are associated. In other words, in the particular embodiment shown, each fluid media reservoir 924 is configured for providing fluid media to a single embryo well 914. As mentioned previously, however, the embryo support device may be configured such that one or more common fluid media reservoirs are provided for multiple embryo wells.

As best seen in FIG. 28, an opening 926 is provided at the base of each fluid media reservoir 925, and is located and configured such that, when second substrate 906 is alignably positioned atop first substrate 905, opening 926 will provide fluid communication between the interior of fluid media reservoir 924 and fluid media input channel 925. In this manner, a plurality of fluid media reservoirs may be distributed circumferentially around each embryo well 914, in fluid communication therewith.

A diaphragm member 928 may be positioned over each fluid media reservoir 924 in order to sealingly enclose each of the fluid media reservoirs 924. In fact, diaphragm members 928 may even allow for the fluid media within reservoirs 924 to be pressurized, as described previously herein. As described above, pressurization of the fluid media may be employed to assist in delivering fluid media to the embryo well.

An input well 930 for each embryo well 914 may also be provided in second substrate 906, as shown. Input well 930 is configured such that when second substrate 906 is alignably positioned on top of first substrate 905, each input well 930 will be in fluid communication with an embryo well 914 through input channel 936. Input well 930 may be used to insert an oocyte, sperm and/or an embryo into well 914. An input cap 931 may also be provided in order to enclose each input well 930, as shown. Although input cap 931 may simply be removed from input well 930 in order to insert an oocyte, sperm or an embryo therein, an input aperture or hole 932 may also be provided in input cap 931. In this manner, an oocyte, sperm or an embryo may be inserted into input well 930 through input hole 932.

Typically, a fluid will be inserted into input well 930 along with an oocyte, sperm or embryo. Thus, the force of gravity alone may cause the oocyte, sperm or embryo to pass from input well 930 through input channel 936 into embryo well 914. Alternatively, input cap 931 may be formed from a resilient or flexible material such that a force may be applied to input cap 931, as shown in FIG. 28 (such as by pressing down on input cap 931 with a finger). Applying such a force to input cap 931 will urge an oocyte, sperm or an embryo from input well 930 into embryo well 914, and will also cause aperture 932 to close.

As shown in the exploded view of FIG. 29, the upper end of each horizontally-oriented embryo well 914 is generally open. However, the embryo well will be substantially sealed once second substrate 906 is alignably secured on top of first substrate 905. In addition, a transparent window 971 (which may, for example, comprise one or more lens elements or simply an optically correct transparent material) is located in second substrate 906 so that window 971 is aligned with embryo well 914. In fact, window 971 may even form part or all of the upper wall of embryo wall 914, as shown. As best seen in the exploded view of FIG. 29, a window 971 is provided for each embryo well 914, and is configured to allow for visualization of an embryo located within each embryo well 914. In the particular configuration, window 971 is positioned within a bore 972 provided in second substrate 906.

Second layer 902 of embryo support device 900 may be reusable in nature, and is primarily designed to accommodate the imaging devices used to acquire image data of the embryos. While second substrate 906 is generally secured or affixed to first substrate 905 (such as by glueing or welding), second layer 902 is preferably removably securable to lower layer 901 (as described previously). For example, upper layer 902 would generally need to be removed in order to load the fluid reservoirs with fluid media, as well as to insert an embryo or other material into input well 930. In fact, as best seen in FIG. 28, upper layer 902 may include interior cavities 991 which are sized and configured to accommodate input caps 931 therein.

Upper layer 902 is also configured to accommodate one or more imaging devices comprising, for example, an imager housing 979 which accommodates an imager 978 (such as a CCD or CMOS imager) and one or more lens elements 977. One or more chambers 980 may be provided in upper layer 902, wherein chambers 980 are sized and configured to accommodate the individual imaging devices (i.e, imager housing 979). Chambers 980 should be located such that when an imaging device is inserted therein, imager 978 may acquire an image of an embryo located within one of the embryo wells 914. In particular, an imaging device may be provided for each of the embryo wells 914, as shown. Each imaging housing 979 may be securely affixed to upper layer 902, or may be removably securable thereto. In this manner, upper layer 902 may even be configured for a single use, while the individual imaging devices may be removed therefrom and reused.

As mentioned previously, one or more additional components described previously herein may also be included in the device of FIGS. 25-30, however these components have not been shown for purposes of clarity. For example, any or all of the various electronic components described previously may be incorporated into embryo support device 900. In fact, FIG. 31 is a schematic illustration of embryo support device 900 incorporating these additional components. It should be pointed out, however, that FIG. 31 does not depict the imaging devices nor light sources which may be provided in embryo support device 900 for purposes of imaging. For example, a light source may be provided for each embryo well 914 in order to facilitate imaging of an embryo located therein. In addition, FIG. 31 only depicts one of embryo wells 914 and the associated fluid media reservoirs 924A-C, input well 930, and waste fluid media chamber 962. Furthermore, transfer sleeve 985 and the associated transfer catheter are also not depicted in FIG. 31 for purposes of clarity.

As shown in the schematic view of FIG. 31, fluid media reservoirs 924 may be loaded with a variety of fluid medias, as desired. In the particular embodiment shown, embryo support device 900 is configured for fertilization of an oocyte as well as the growth of the resulting embryo. Therefore, reservoir 924A is loaded with an enzyme solution for bathing the oocyte prior to fertilization. The enzyme may, for example, be chosen to break up the cumulus cells surrounding the oocyte, and may comprise, for example, hyaluronidase. Fluid media reservoir 924B may be loaded with a wash fluid used to wash the embryo after fertilization (e.g., to remove cumulus cells and the enzyme). Finally, fluid media reservoir 924C is depicted as being loaded with a fluid growth media suitable for embryo development. FIG. 31 also depicts sperm being loaded into input well 930, as well as an optional control valve 945 provided along input channel 936 for controlling the delivery of sperm or other material from input well 930 to embryo well 914. It should also be pointed out that while FIG. 31 depicts an embryo E located within well 914, prior to fertilization an oocyte will be located therein rather than an embryo.

An oocyte is inserted into culturing well 914, such as by means of input well 930. A suitable fluid may also be loaded into well 914 along with the oocyte. Thereafter, piezoelectric element 257, or other acoustic-wave generating device, may be activated in order to remove cumulus cells and other debris from the oocyte, in the manner described previously. In the meantime, sperm may be loaded into input well 930, as shown. It is also contemplated that one or more additional fluid media reservoirs may be provided, such that a sperm may be loaded into a fluid media reservoir rather than input well 930.

After the cumulus cells have dislodged from the oocyte, the oocyte may be flushed with the enzyme solution from reservoir 924A by, for example, activating control valve 927. The enzyme solution will force the cumulus cells and other debris out of culturing well 914, through filter 956 into waste media reservoir 962. A valve 946 may also be provided along waste fluid channel 963, as shown, in order to control the flow of fluid through channel 963. After flushing, the sperm solution in input well 930 may be delivered to culturing well 914 through input channel 936. Although a valve 945 may be provided on input channel 936, the sperm may also be injected manually into culturing well 914, such as by using flexible input cap 931 (as described previously).

After a period of time suitable for fertilization, and/or after fertilization has occurred (as determined, for example, by the imaging system of device 900), a wash fluid may be urged from reservoir 924B into culturing well 914. The wash fluid will thus urge sperm and other debris through filter 956 into waste fluid media reservoir 962. Thereafter, fluid growth media may be urged from reservoir 924C into embryo well 914 so as to provide a suitable fluid media for further development of the embryo.

As described previously herein, all of the above processes can be controlled by CPU 296, or other suitable processors or controllers in device 900. In general, CPU 296 will control these processes according to one or more instruction sets stored in memory 297. CPU 296 may control, for example, the operation of heater 254, the various fluid control valves described previously, pH adjustment devices, imaging devices, and/or the various other componentry described previously herein. A display 280 may also be provided on embryo support device 900 in any convenient location thereon. Likewise, one or more interfaces 289 may also be provided so that device 900 may be placed in communication with one or more external devices.

As described previously, embryo support device 900 may be configured to be completely self-contained. It is also contemplated that, like cartridge 300, embryo support device 900 may also be configured for insertion into a base station, as described previously. Although such a base station would generally not include an imaging device or a motor for causing rotation or other movement of embryo support device 900, such a base station may include the other componentry described previously for base unit 315. For example, rather than providing a display device on each embryo support device 900, a common display device may be provided on a base station to which embryo support device 900 may be operatively connected or attached. It should be kept in mind, however, that such connection may even be accomplished through one or more wireless interfaces, as described above. In this manner, data (including, for example, data acquired by the various sensors and the imaging devices) may be transmitted to a base assembly and/or other external device (such as an external computing device). Data stored in memory 297 may be transmitted in this manner, as well as data acquired in “real-time”. Information may also flow in the opposite direction in that interface 289 of embryo support device 900 may be used to receive data from an external device, such as new or revised instruction sets. For example, since device 900 will generally use one or more feedback or feedforward control systems for regulating one or more of the conditions within well 914, the instructions received by device 900 may include, for example, revised control parameters for such control systems.

It should also be noted that although the present invention has been described in conjunction with growth media, wash fluids or enzyme solutions located within the various fluid media reservoirs described herein, various other types of fluids may be provided therein for delivery to an embryo (such as one or more drug solutions). Alternatively, in the embodiment of FIGS. 25-30, such additional fluids may be delivered to an embryo, as needed, using input wells 930.

It should be pointed out that the above description of the embryo support devices, systems and methods according to various embodiments of the present invention are merely exemplary. For example, any number of fluid media reservoirs may be provided, including reservoirs containing more than one distinct fluid media. Accordingly, the scope of the present invention should be considered in terms of the following claims, and it is understood not to be limited to the details of the structure and operation shown and described in the specification and the drawings.

What is claimed is:

1. A self-contained embryo support assembly, comprising:
(a) a well for housing an embryo and a fluid therein;
(b) a control system for regulating one or more conditions within 5 said well; and
(c) an energy source;
wherein said energy source is configured for powering said control system without connecting said embryonic support assembly to an external power source.

2. The embryonic support assembly of claim 1, wherein said control system comprises a heater configured for regulating the temperature within said well.

3. The embryonic support assembly of claim 2, wherein said control system further comprises a processor, and at least one temperature sensor in electrical communication with said processor, wherein said processor is configured for regulating said heater.

4. The embryonic support assembly of claim 1, wherein said control system comprises at least one sensor chosen from the group consisting of a pH sensor, an oxygen sensor, a carbon dioxide sensor, an urea sensor, an ammonia sensor, a nitrogen sensor, a calcium ion sensor, a sodium ion sensor, a nitrate sensor, a phosphate sensor and an osmolarity sensor.

5. The embryonic support assembly of claim 1, further comprising a fluid supply system configured for delivering fluid media to said well.

6. The embryonic support assembly of claim 5, wherein said fluid supply system comprises a fluid reservoir in fluid communication with said well.

7. The embryonic support assembly of claim 6, wherein said fluid reservoir is pressurizable so that the fluid reservoir may be filled with a fluid media under pressure for delivery to said well.

8. The embryonic support assembly of claim 7, wherein said fluid supply system further comprises a valve for controlling the delivery of fluid media from said fluid reservoir to said well.

9. The embryonic support assembly of claim 6, wherein said fluid supply system further comprises a pump for delivering fluid media from said fluid reservoir to said well.

10. The embryonic support assembly of claim 6, wherein said control system is configured for regulating the delivery of fluid media from said fluid reservoir to said well.

11. The embryonic support assembly of claim 5, further comprising a waste fluid reservoir in fluid communication with said well.

12. The embryonic support assembly of claim 1, wherein said control system is configured for regulating one or more conditions within said well according to a predetermined set of instructions.

13. The embryonic support assembly of claim 11, wherein said assembly is configured such that said predetermined set of instructions may be replaced or altered in response to a signal received from another device.

14. The embryonic support assembly of claim 1, further comprising an identification means for identifying the embryonic support assembly or an embryo located therein, said identification means chosen from the group consisting of: printed indicia, memory, an etched indicia, an engraved indicia, a barcode, and radioactive tagging.

15. The embryonic support assembly of claim 1, further comprising a memory, wherein said assembly is configured for periodically acquiring data concerning one or more conditions within said well and storing said data in said memory.

16. The embryonic support assembly of claim 1, wherein said well has been formed by at least one of micromachining, microembossing and micromolding of a substrate.

17. The embryonic support assembly of claim 1, wherein said well is configured such that an embryo located therein will tend to remain at a predetermined location within said well.

18. The embryonic support assembly of claim 17, wherein said well is has a tapered configuration such that an embryo located therein will be directed to said predetermined location within said well.

19. The embryo support assembly of claim 1, wherein said assembly comprises a MEMS device.

20. A method for shipping a metabolically active embryo, comprising the steps of:
(a) providing a self-contained embryonic support assembly having a well housing an embryo and a fluid therein, and a control system for regulating one or more conditions within said well during shipment; and
(b) causing said embryonic support assembly to be transported from one location to another.

21. The method of claim 20, wherein said embryonic support assembly further comprises an energy source, wherein said energy source is configured for powering said control system without connecting said embryonic support assembly to an external power source.

22. The embryonic support assembly of claim 20, wherein said control system comprises a heater configured for regulating the temperature within said well.

23. The embryonic support assembly of claim 22, wherein said control system further comprises a processor, and at least one temperature sensor in electrical communication with said processor, wherein said processor is configured for regulating said the temperature within said well.

24. An embryo support assembly, comprising:
(a) a well for housing an embryo and a fluid therein; and
(b) a plurality of stations, each of which is configured for communication with said well;
wherein each of said plurality of stations are configured such that they may be selectively brought into communication with said well.

25. The embryo support assembly of claim 24, wherein at least one of said well and said plurality of stations are selectively moveable with respect to one another such that each of said plurality of stations may be selectively brought into communication with said well

26. The embryo support assembly of claim 25, wherein at least one of said plurality of stations comprises a fluid reservoir for supplying fluid media to said well.

27. The embryo support assembly of claim 26, wherein said plurality of stations are configured for rotational movement with respect to said well.

28. The embryo support assembly of claim 27, wherein said plurality of stations are configured for rotational movement around at least a portion of said well.

29. The embryo support assembly of claim 26, wherein said plurality of stations are arranged linearly adjacent said well.

30. The embryo support assembly of claim 25, wherein at least one of said stations comprises a passageway through which an embryo can be inserted into or removed from said well when said passageway is in communication with said well.

31. The embryo support assembly of claim 25, wherein at least one of said stations comprises a fertilization passageway sized and configured to allow the insertion of a fertilization device for injecting a sperm into an egg located within said well when said fertilization passageway is in communication with said well.

32. The embryo support assembly of claim 25, wherein at least one of said stations comprises a visualization station.

33. An embryo support assembly, comprising:
(a) at least two wells for housing an embryo and a fluid therein, said wells arranged vertically above one another; and
(b) at least one passageway which provides communication between said wells;
wherein said embryo support assembly is configured such that an embryo located within the uppermost well will travel downwardly through said at least one passageway into the second well under the action of gravity.

34. The embryo support assembly of claim 33, wherein said assembly is configured for selectively controlling the movement of an embryo from one well to the next.

35. The embryo support assembly of claim 34, wherein said uppermost well is selectively rotatable such that rotation of said uppermost well brings said well into communication with said passageway, thereby allowing an embryo located in said uppermost well to travel downwardly through said passageway.

36. The embryo support assembly of claim 35, wherein said passageway comprises a fluid media reservoir.

37. The embryo support assembly of claim 36, further comprising an upper fluid media reservoir located above said uppermost well.

38. An embryo support system, comprising:
(a) at least one embryo support assembly having a well for housing an embryo and a fluid therein;
(b) a cartridge configured for removably receiving said at least one embryo support assembly;
(c) a base assembly configured for removably receiving said cartridge; and
(d) a control system for regulating one or more conditions within said well.

39. The embryo support system of claim 38, wherein said control system is provided in said embryo support assembly.

40. The embryo support system of claim 38, wherein said cartridge is configured for removably receiving a plurality of embryo support assemblies, and further comprising a plurality of embryo support assemblies.

41. The embryo support system of claim 39, wherein said cartridge has a plurality of chambers, each of which is configured for removably receiving one of said embryo support assemblies.

42. The embryo support system of claim 41, wherein said chambers are arranged in a circular, linear or rectilinear array.

43. The embryo support system of claim 38, further comprising an imaging system configured for acquiring an image of an embryo located within said well.

44. The embryo support system of claim 43, wherein said imaging system is provided in said base assembly.

45. The embryo support system of claim 44, wherein said base assembly further includes a display device configured for displaying images acquired by said imaging system.

46. The embryo support system of claim 43, wherein said imaging system comprises at least one light responsive sensor and a light source.

47. The embryo support system of claim 46:
further comprising a plurality of embryo support assemblies;
wherein said imaging system is provided in said base assembly;
wherein said cartridge is configured for removably receiving said plurality of embryo support assemblies; and
wherein said imaging system is configured for acquiring images of en embryo located within the well of each of said plurality of embryo support assemblies.

48. The embryo support system of claim 38, wherein said system is configured such that the system may be placed in electrical communication with a remote computing device.

49. The embryo support system of claim 48, wherein said system is configured for transmitting data to a remote computing device.

50. The embryo support system of claim 38, wherein said base assembly includes a central processing unit and memory.

51. The embryo support system of claim 39, wherein said base assembly is in electrical communication with said embryo support assembly.

52. An embryo support device, comprising:
(a) at least one embryo support assembly having a well for housing an embryo and a fluid therein;
(b) a cartridge configured for removably receiving said at least one embryo support assembly.

53. The embryo support device of claim 52, wherein said cartridge is configured for removably receiving a plurality of embryo support assemblies, and further comprising a plurality of embryo support assemblies.

54. The embryo support device of claim 53, wherein said cartridge has a plurality of chambers, each of which is configured for removably receiving one of said embryo support assemblies.

55. The embryo support device of claim 54, wherein said chambers are arranged in a circular, linear or rectilinear array.

56. The embryo support device of claim 54, wherein said cartridge further comprises:
at least one chamber configured for removably receiving said at least one embryo support assemby; and
at least one aperture which intersects said chamber; wherein said aperture and said embryo support assembly are configured such that an embryo positioned within said well may be visualized through said aperture.

57. The embryo support device of claim 55, wherein said chambers are arranged circularly, and said cartridge is ring-shaped.

58. The embryo support device of claim 55, wherein said chambers are arranged circularly, and said cartridge is disk-shaped.

59. The embryo support device of claim 52, further comprising an imaging system configured for acquiring an image of an embryo located within said well.

60. The embryo support device of claim 58, wherein said imaging system comprises at least one light responsive sensor and at least one light source.

62. The embryo support device of claim 59, wherein said imaging system is configured such that a portion of said cartridge may be positioned between said light source and said at least one light responsive sensor in order to acquire an image of an embryo located within said well.

63. The embryo support device of claim 60:
wherein said cartridge is configured for removably receiving a plurality of embryo support assemblies;
further comprising a plurality of embryo support assemblies; and
wherein said imaging system is configured such that a portion of said cartridge may be selectively positioned between said light source and said at least one light responsive sensor in order to acquire an image of each embryo located within a well of an embryo support assembly received by said cartridge.

64. An embryo support device, comprising:
(a) a plurality of wells, each configured for housing an embryo and a fluid therein; and
(b) a fluid supply system for each of said wells, each of said fluid supply systems comprising a plurality of fluid reservoirs in fluid communication with the well associated with the fluid supply system.

65. The embryo support device of claim 64, further comprising a control system for regulating one or more conditions within said wells.

66. The embryo support device of claim 64, further comprising a plurality of imaging devices, each of said imaging devices configured for acquiring image data of an embryo located within one of said wells.

67. The embryo support device of claim 64, further comprising a plurality of waste reservoirs, each of said waste reservoirs configured in fluid communication with one of said wells.

68. The embryo support device of claim 65, wherein said control system comprises at least one processor and at least one memory, wherein said control system is configured for regulating one or more conditions within said wells according to at least one set of instructions stored in 5 said memory.

69. The embryo support device of claim 68, wherein said device is configured for acquiring data concerning at least one of the development of one or more embryos positioned within said wells, and the conditions within said wells.

70. The embryo support device of claim 68, wherein said device is further configured such that said for performing at least one of storing said data in said memory, and transmitting said data to another device.

71. The embryo support device of claim 68, wherein said device is further configured such that said at least one set of instructions may be replaced or altered in response to a signal received from another device.

72. The embryo support device of claim 64, wherein said device is substantially disc-shaped, and said wells are arranged in a substantially circular array.
